(19) United States
(12) Patent Application Publication
(10) Pub. No.: US 2026/0232575 A1
(43) Pub. Date: Aug. 13, 2026

(54) COMPOSITIONS AND TECHNIQUES FOR TREATMENT OF POLYCYSTIC OVARIAN SYNDROME (PCOS) AND ENDOMETRIOSIS

(71) Applicant: Daré Bioscience, Inc., San Diego, CA (US)

(72) Inventors: Sabrina Martucci Johnson, San Diego, CA (US); David Friend, San Diego, CA (US); Nicolas J. Pacelli, Cedar Park, TX (US)

(21) Appl. No.: 19/149,689

(22) PCT Filed: Jan. 18, 2024
(86) PCT No.: PCT/US24/11949

§ 371 Date: Jul. 21, 2025

Related U.S. Application Data
(60) Provisional application No. 63/480,582, filed on Jan. 19, 2023.

(51) Int. Cl. A61K 9/00; A61K 31/4045; A61K 47/10; A61K 47/32

(52) U.S. Cl. CPC A61K 9/0036; A61K 31/4045; A61K 47/10; A61K 47/32

(57) ABSTRACT

The present disclosure generally relates to compositions and methods for treating polycystic ovarian syndrome (PCOS) and/or endometriosis. The composition may contain an active ingredient for treatment of PCOS and/or endometriosis, e.g., melatonin, a melatonin receptor agonist, and/or salts thereof. In some embodiments, a composition such as a gel may be applied to the vagina of a subject that is relatively viscous, for example, with a viscosity at room temperature of at least 1.5 million cP. Compositions having such relatively high viscosities may be useful, for example, to prevent the composition from readily exiting the vagina or degrading too quickly. In some embodiments, a drug delivery device containing the active agent may be inserted in the vagina. In some embodiments, the drug delivery device is an intravaginal ring.

### RELATED APPLICATIONS

This application claims the benefit of priority under 35 U.S.C. § 119(e) to U.S. Provisional Application No. 63/480,582, filed Jan. 19, 2023, the entire contents of which is incorporated herein by reference in its entirety.

### FIELD

The present disclosure generally relates to compositions and methods for the treatment of polycystic ovarian syndrome (PCOS) and endometriosis using melatonin or a melatonin receptor agonist.

### BACKGROUND

A number of gynecological diseases remain difficult to diagnose and treat. Polycystic ovarian syndrome (PCOS) is a disease occurring in females, and is characterized by hyperandrogenism. Current treatment strategies for PCOS have limited efficacy and marked side effects. Endometriosis is one of the most common gynecological diseases in the United States. It is a painful, often debilitating disease that affects more than 6.5 million women in America between the ages of 15 and 44. The cause of endometriosis is unknown. Possible causes include retrograde menstrual bleeding, genetic factors, immune system problems, hormonal imbalance, and surgical complications. Because the cause of endometriosis is not known, current treatments for endometriosis only treat the symptoms, not the disease itself.

There exists a need for new therapies for the treatment of PCOS and endometriosis.

### SUMMARY

The present disclosure generally relates to compositions and methods for treating PCOS and/or endometriosis. The subject matter of the present disclosure involves, in some cases, interrelated products, alternative solutions to a particular problem, and/or a plurality of different uses of one or more systems and/or articles.

In one aspect, the present disclosure is directed to a method for treating PCOS by administration of melatonin, a melatonin receptor agonist, or a pharmaceutically acceptable salt thereof to a subject.

In another aspect, the present disclosure is directed to a method for treating endometriosis by administration of melatonin, a melatonin receptor agonist, or a pharmaceutically acceptable salt thereof to a subject.

In some embodiments, the melatonin, a melatonin receptor agonist, or pharmaceutically acceptable salt thereof is administered to the vagina of the subject (e.g., intravaginally). In some embodiments, the melatonin, a melatonin receptor agonist, or pharmaceutically acceptable salt thereof is in a gel formulation. In some embodiments, the melatonin, a melatonin receptor agonist, or pharmaceutically acceptable salt thereof is in a drug delivery device. In some embodiments, the drug delivery device is an intravaginal ring.

In another aspect, the present disclosure is directed to a composition including melatonin or a melatonin receptor agonist, as an active agent, and methods of using the same. The composition, in one set of embodiments, comprises a poloxamer and a stabilization polymer. The composition may have a viscosity at room temperature of at least 1.5 million cP.

According to another set of embodiments, the composition comprises a poloxamer, and a stabilization polymer. The composition may be made by a process comprising forming a composition comprising the poloxamer and the stabilization polymer, and removing air from the composition.

The composition, in another set of embodiments, comprises a poloxamer and a stabilization polymer. In some embodiments, the composition is made by a process comprising forming a composition comprising the poloxamer and the stabilization polymer, and exposing the composition to a pressure of less than 100 mbar (absolute) for at least 30 minutes.

In one set of embodiments, the composition comprises a poloxamer, a stabilization polymer, and an active ingredient for treating polycystic ovarian syndrome (PCOS) and/or treating endometriosis. In some cases, the composition has a viscosity at room temperature of at least 1.5 million cP.

In another set of embodiments, the composition comprises a poloxamer, a stabilization polymer, and an active ingredient for treating PCOS and/or treating endometriosis. In some cases, the composition is made by a process comprising forming a composition comprising the poloxamer, the stabilization polymer, and the active ingredient (e.g., melatonin or a melatonin receptor agonist), and removing air from the composition.

In yet another set of embodiments, the composition comprises a poloxamer, a stabilization polymer, and an active ingredient for treating PCOS and/or treating endometriosis. In some cases, the composition is made by a process comprising forming a composition comprising the poloxamer, the stabilization polymer, and the active ingredient (e.g., melatonin or a melatonin receptor agonist), and exposing the composition to a pressure of less than 100 mbar (absolute) for at least 30 minutes.

In still another set of embodiments, the composition is a composition for treating PCOS and/or treating endometriosis. In some cases, at least 90 wt % of the composition consists essentially of a poloxamer, a stabilization polymer, an active ingredient for treating PCOS and/or treating endometriosis (e.g., melatonin or a melatonin receptor agonist), and water.

In another aspect, the present disclosure is directed to a method. The method, according to one set of embodiments, comprises applying, to the vagina of a subject, a gel having a viscosity at room temperature of at least 1.5 million cP.

In another set of embodiments, the method comprises applying, to a vagina of a subject, a composition comprising a poloxamer and a stabilization polymer. In certain embodiments, the composition, as applied, has a viscosity of at least 1.5 million cP.

In another set of embodiments, the method comprises providing a composition comprising a poloxamer and a stabilization polymer, and removing air from the composition.

The method, in still another set of embodiments, comprises providing a composition comprising a poloxamer and a stabilization polymer, and exposing the composition to a pressure of less than 100 mbar (absolute) for at least 30 minutes to form a gel.

In one set of embodiments, the method comprises applying, to a vagina of a subject, a composition comprising a poloxamer, a stabilization polymer, and an active ingredient for treating PCOS and/or treating endometriosis (e.g., melatonin or a melatonin receptor agonist). In some cases, the composition, as applied, has a viscosity of at least 1.5 million cP.

In another set of embodiments, the method comprises providing a composition comprising a poloxamer, a stabilization polymer, and an active ingredient for treating PCOS and/or treating endometriosis (e.g., melatonin or a melatonin receptor agonist), and removing air from the composition.

In yet another set of embodiments, the method comprises providing a composition comprising a poloxamer, a stabilization polymer, and an active ingredient for treating PCOS and/or treating endometriosis (e.g., melatonin or a melatonin receptor agonist), and exposing the composition to a pressure of less than 100 mbar (absolute) for at least 30 minutes to form a gel.

In another set of embodiments, the method comprises providing a composition comprising a poloxamer, a stabilization polymer, and an active ingredient for treating for treating PCOS and/or treating endometriosis (e.g., melatonin or a melatonin receptor agonist), and removing air from the composition.

In yet another set of embodiments, the method comprises providing a composition comprising a poloxamer, a stabilization polymer, and an active ingredient for treating for treating PCOS and/or treating endometriosis (e.g., melatonin or a melatonin receptor agonist), and exposing the composition to a pressure of less than 100 mbar (absolute) for at least 30 minutes to form a gel.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, a composition as described herein. In still another aspect, the present disclosure encompasses methods of using one or more of the embodiments described herein, for example, a composition for the treatment of as described herein, and other indications.

In some embodiments, the present disclosure provides kits comprising the compositions described herein, where the kit includes an applicator suitable for vaginal application. In some embodiments, the applicator is pre-filled with the compositions described herein. In some embodiments, the applicator is not pre-filled with the compositions described herein.

In some embodiments, the kit includes one or more of instructions for inserting the applicator into the vagina and instructions for treating PCOS and/or treating endometriosis (e.g., melatonin or a melatonin receptor agonist) by applying the applicator filled with the composition or gel.

In some embodiments, the composition is at a temperature of about 4° C. for pre-filling the applicator.

In aspects, provided herein is an implantable drug delivery device including: an inner core having at least one active pharmaceutical ingredient distributed throughout a first water-insoluble polymer, wherein the at least one active pharmaceutical ingredient includes melatonin or a melatonin receptor agonist; an intermediate coating positioned around the inner core, said intermediate coating comprising an acrylate polymer; and an outer coating positioned around the intermediate coating, said outer coating comprising a second water-insoluble polymer; wherein the acrylate polymer is formed from one or more monomers of formula (I):

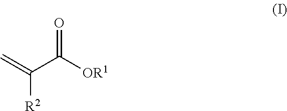

wherein R¹ is selected from H, alkyl, alkenyl, alkynyl, or aryl; and R² is selected from H or alkyl.

In embodiments, the device is an intravaginal ring or a subcutaneous implant. In some embodiments, the total amount of the at least one pharmaceutical ingredient is at least 2 wt %, preferably at least 5 wt %, based on the total weight of the inner core. In yet other embodiments, total amount of the at least one pharmaceutical ingredient is less than 50 wt %, preferably less than 30 wt %, based on the total weight of the inner core.

In embodiments, the device is used for treating PCOS. In other embodiments, the device is used for treating endometriosis.

In some embodiments, the active ingredient (e.g., melatonin or a melatonin receptor agonist) is administered via a segmented, e.g., an ethylene-vinyl-acetate (EVA), intravaginal ring (IVR). In some embodiments, EVA-based segmented intravaginal rings (IVRs) are designed to release about 4 to about 12 mg progesterone (P) per day over days 2-14. In an aspect of the disclosure, the disclosed EVA rings are used to treat PCOS in a subject. In another aspect, the disclosed EVA rings are used to treat endometriosis in a subject.

In some embodiments, a rate of release of melatonin or melatonin receptor agonist is in the range of 1 mg/day to about 1000 mg/day which includes ranges that fall in between the 1 mg/day to about 1000 mg/day range.

An aspect of the disclosure is an ethylene-vinyl-acetate (EVA), intravaginal ring (IVR), where the ring contains at least two segments/fibers, wherein one segment contains melatonin or melatonin receptor agonist. In an embodiment of this aspect, the ring is about 57 mm in diameter with a cross-section diameter of about 5 mm.

In some embodiments of the EVA ring, the EVA segment containing melatonin or melatonin receptor agonist is about 74.5 mm to about 156.0 mm in length or about 74.5 mm to about 148.5 mm in length. In some embodiments of the EVA ring, the length of the EVA segment containing melatonin or melatonin receptor agonist is selected from a size of about 74.5 mm, about 148.5 mm and about 156.0 mm.

In some embodiments, the EVA rings are used to treat PCOS. In other embodiments, the EVA rings are used to treat endometriosis.

Other advantages and novel features of the present disclosure will become apparent from the following detailed description of various non-limiting embodiments of the disclosure when considered in conjunction with the accompanying figures.

### DETAILED DESCRIPTION

The present disclosure generally relates to compositions and methods for treating PCOS and/or endometriosis.

In some embodiments, a composition such as a gel may be applied to the vagina of a subject that is relatively viscous, for example, with a viscosity at room temperature of at least 1.5 million cP. The composition contains an active ingredient for treatment of PCOS and/or endometriosis, e.g., melatonin or a melatonin receptor agonist, and/or salts thereof.

Compositions having such relatively high viscosities may be useful, for example, to prevent the composition from readily exiting the vagina or degrading too quickly. This may, for example, allow the composition to release the active ingredient over a relatively long period of time to the vagina. In some embodiments, such compositions may be prepared by removing air from the composition to increase its viscosity or cause the composition to form a gel, etc. In addition, certain embodiments as described herein are generally directed to techniques for making or using such compositions, kits including such compositions, or the like.

Contrary to compositions for dermal or surface applications, the compositions described herein are formulated for vaginal application (e.g., a mucosal surface). The vaginal epithelium has completely different biophysical properties compared to other skin surfaces. For example, the inner lining of the vagina consists of multiple layers of (squamous) cells. The basal membrane provides the support for the first layer of the epithelium-the basal layer. The intermediate layers lie upon the basal layer, and the superficial layer is the outermost layer of the epithelium. In developing vaginal compositions, additional factors are considered, including viscosity. This is because materials applied to the vagina may be expelled relatively quickly (e.g., due to gravity or vaginal discharge) and thus the viscosity (thickness or stickiness) of the vaginal composition is particularly relevant. Also, the mucosal layer within the vagina presents a unique challenge for drug entry, and the vagina is designed to permit the passage of normal uterine secretions (e.g., flushing of the vaginal discharge). In light of these challenges, developing vaginal compositions presents itself with unique hurdles as compared to developing compositions for other dermal uses. Moreover, advantages of preparing and formulating compositions for vaginal (local) delivery include lower systemic side effects due to lower systemic concentrations of the drug, but equivalent or better symptom relief due to local delivery of the drug to the affected tissue.

### Compositions

One aspect as discussed herein is generally directed to compositions having relatively high viscosities, or resistance to flow or deformation. In some embodiments, such compositions may have viscosities of, for example, at least 1 million cP, 3 million cP, or more, or other viscosities as discussed in more detail below. Fluids with such relatively high viscosities do not flow easily and may even resist flow due to gravity in some cases.

The compositions may contain a polymer, which can increase the viscosity of the composition. For example, the polymer may include a poloxamer, which may form a gel in some cases. In addition, in some cases, the composition may also contain xanthan gum, e.g., that can also act as a stabilizer or a thickening agent, which may help increase viscosity. The composition may also contain an active ingredient, such as melatonin or a melatonin receptor agonist, which can be released from the composition over a suitable period of time. The composition may be applied to the vagina or another suitable body cavity of a subject, for example, where release of the active ingredient is desired, e.g., to treat PCOS or to treat endometriosis. More details of these and other compositions, in accordance with various embodiments, are provided below.

Such compositions containing relatively high viscosities may be particularly useful, in certain embodiments, to prevent the composition from readily exiting the vagina (or other cavity), and/or from degrading too quickly after application. This may be used, for example, to assist the delivery of an active ingredient to the subject, e.g., to the vagina of the subject. For instance, fluids with relatively high viscosities may release the active ingredient more slowly and/or uniformly, thereby allowing the active ingredient to be delivered to the subject over a longer period of time. Higher viscosities may also better resist the natural function of the vagina to discharge. Thus, as discussed in more detail herein, the active ingredient may be delivered, e.g., at suitably effective concentrations or amounts, for example, over a period of at least a day, a week, or even longer in some cases. In addition, in some embodiments, only a single dose of the composition may be required to treat a subject, e.g., since the composition does not readily exit the vagina.

In some cases, the composition may be inserted into the vagina, and an active ingredient such as melatonin or a melatonin receptor agonist may be delivered, e.g., to the vagina. This may be useful, for example, to treat PCOS and/or to treat endometriosis. The composition may be applied to the vagina of the subject, and an active ingredient, such as melatonin or a melatonin receptor agonist, may be delivered to the vagina.

Although other techniques for delivering active ingredients to the vagina (or other body cavity) may also involve the use of relatively high viscosities fluids, these fluids are often selected to have lower viscosities at room temperatures (e.g., about 25° C.) and higher viscosities at body temperatures (e.g., 37° C.). For example, they may be a liquid at room temperature, but become a gel at body temperature. In addition, they may not have viscosities as high as 1 million cP or more; as an example, such a composition may have a viscosity of 300,000 cP at room temperature (and be relatively flowable), increasing to only about 800,000 cP at body temperature.

In contrast, the compositions as discussed herein may have relatively high viscosities, for instance, viscosities as high as 1 million cP, 1.5 million cP or more, even at room temperature. It should be noted that such compositions, due to their high viscosities, are actually difficult to manufacture; accordingly, most other techniques will use compositions with either lower viscosities, or viscosities that are at least low at room temperatures, typically well below 1 million cP. However, without wishing to be bound by any theory, it is believed that having a high initial viscosity, e.g., upon application to the vagina, may be surprisingly useful for rapid symptom relief A composition with a relatively high viscosity is less able to lose the active ingredient, i.e., prior to application to the vagina, and thus retains the active ingredient for release to the subject more readily than a liquid or less viscous composition. For example, the composition may at least partially seal the vagina better, e.g., to promote treatment with the active ingredient. In addition, a composition with a relatively high viscosity may itself also stay within the vagina longer. Accordingly, such compositions may produce better symptom resolution, e.g., as compared to formulations with relatively low viscosities at room temperature.

Such high viscosities can be achieved, in various embodiments, using techniques such as removing air from the composition, which may increase its viscosity and/or cause it to form a gel. Other techniques may also be used, including any of those described herein. For example, in certain embodiments, a composition may be prepared, e.g., comprising poloxamer, an active ingredient, xanthan gum, water, etc., and the composition treated to remove air from the composition, for example, to reduce the composition to 15 vol % air, or less. Techniques for removing air include, but are not limited to, a variety of techniques, such as centrifugation or exposure to relatively high vacuums, e.g., less than 100 mbar.

In addition, it should be understood that the composition need not be limited to those described above, and in other embodiments, other compositions may also be used. Any composition suitable for administration to the vagina may be used in the methods and compositions described herein. Non-limiting examples of compositions can be found in PCT Patent Application No. PCT/US2022/038234, and U.S. Pat. Nos. 8,691,278; 8,501,230; 11,129,896; 10,238,663; and 10,874,612, each of which is incorporated herein by reference in its entirety, for all compositions, methods of making, methods of using, formulations, and the like taught therein. Additional non-limiting examples of such compositions follow.

### Poloxamer

For instance, in some aspects, a composition may include one or more poloxamers, xanthan gum, and/or another stabilization polymer, and an active ingredient such as any of those discussed herein. Water may be present in some cases, e.g., such that the composition is a gel, and/or has a relatively high viscosity at room temperature, such as is described herein. Other components may be present as well in certain embodiments, for instance, citrate and/or a citrate salt, benzyl alcohol, or the like. These may act, for example, as excipients, preservatives, antimicrobials, bulking agents, stabilizers, antioxidants, buffers, pH regulating agents, or the like. In addition, in some cases, other components that increase the viscosity of the composition may also be used, for example, hyaluronic acid, alginic acid, modified celluloses such as hydroxypropyl methylcellulose, in addition or instead of poloxamer.

As mentioned, the composition may include one or more poloxamers in one set of embodiments. The poloxamer may be used to increase the viscosity of the composition, e.g., as described herein. In some cases, sufficient poloxamer may be present to cause the composition to form a gel, e.g., at room temperature (about 25° C.) and/or body temperature (about 37° C.).

Furthermore, in some cases, the composition may have a gelling temperature, but the gel temperature may be in a range that is physiologically irrelevant. For instance, the gelling temperature may be above 40° C., or below 25° C., and thus, the composition does not change phase or gel at normal physiological or body temperatures.

Poloxamers generally include any of a variety of polyoxyethylene-polyoxypropylene triblock copolymers. In some cases, the poloxamer may be a nonionic block copolymer composed of a central hydrophobic chain of polyoxypropylene (polypropylene oxide) flanked by two hydrophilic chains of polyoxyethylene (polyethylene oxide). In some embodiments, the poloxamers may be soluble in water and other polar and non-polar solvents.

Because the lengths of the polymer blocks can be independently customized, many different poloxamers exist that have slightly different properties. For example, the poloxamer may have a structure:

HO—[CH₂—CH₂—O]ₐ—[CH₂—CH(CH₃)—O]_{b}—[CH₂—CH₂—O]ₐ—H.

The structure includes a core of propylene oxide (represented by “b” in the above figure), flanked by ethylene oxide subunits (represented by “a” in the above figure), typically on both sides. The sum of the two a's may be, for example, from 10 to 1000, from 50 to 500, from 100 to 300, from 150 to 250, or 200. As another example, a may be between 99 and 103, e.g., 101. In embodiments, b may be, for example, from 30 to 100, from 50 to 80, from 60 to 70, or 65. As another example, b may be between 54 and 58, e.g., 56.

In some embodiments, the ethylene oxide subunits forming the poloxamer may be in molar excess to the propylene oxide subunits. For example, in certain embodiments, the ratio of ethylene oxide subunits to propylene oxide subunits (i.e., a:b) may be, for example, from 1.1:1 to 8:1, from 3:1 to 5:1, or from 2:1 to 4:1.

Several suitable poloxamers can be readily obtained commercially, including poloxamer 407, Pluronic® F-127, or the like. The composition may include a single poloxamer, or more than one type of poloxamer. In some cases, at least 30 wt %, at least 50 wt %, at least 60 wt %, at least 70 wt %, at least 80 wt %, or at least 90 wt % of the poloxamer within the composition is a single type of poloxamer, for example, poloxamer 407 or Pluronic® F-127.

The molecular weight of the poloxamer may be, in one embodiment, from 5 kDa to 25 kDa. In some instances, the molecular weight of the copolymer may be from 9 kDa to 16 kDa. In some cases, the molecular weight of the poloxamer may be at least 1 kDa, at least 2 kDa, at least 3 kDa, at least 4 kDa, at least 5 kDa, at least 7 kDa, at least 9 kDa, at least 10 kDa, at least 15 kDa, at least 16 kDa, at least 20 kDa, at least 25 kDa, at least 50 kDa, etc. In addition, in certain embodiments, the molecular weight of the poloxamer may be no more than 50 kDa, no more than 25 kDa, no more than 20 kDa, no more than 16 kDa, no more than 15 kDa, no more than 10 kDa, no more than 9 kDa, no more than 5 kDa, no more than 4 kDa, no more than 3 kDa, no more than 2 kDa, no more than 1 kDa, etc. Combinations of any of these are also possible. For instance, the poloxamer may have a molecular weight of between 10 kDa and 15 kDa. As other non-limiting examples, the molecular weight may be between 3 kDa and 5 kDa, between 2 kDa and 4 kDa, between 5 kDa and 20 kDa, between 9 kDa and 16 kDa, etc. The molecular weight, in some cases, may be determined as a weight average molecular weight.

In certain embodiments, the poloxamer may be present within the composition at concentrations of at least 1 wt %, at least 2 wt %, at least 3 wt %, at least 5 wt %, at least 7 wt %, at least 10 wt %, at least 15 wt %, at least 20 wt %, at least 25 wt %, at least 30 wt %, at least 40 wt %, or at least 50 wt %. In addition, in some embodiments, the poloxamer may be present within the composition at concentrations of no more than 50 wt %, no more than 40 wt %, no more than 30 wt %, no more than 25 wt %, no more than 20 wt %, no more than 15 wt %, no more than 10 wt %, no more than 7 wt %, no more than 5 wt %, no more than 3 wt %, no more than 2 wt %, etc. Combinations of any of these are also possible in other embodiments. For example, the poloxamer may be present in a composition at between 10 wt % and 20 wt %, between 5 wt % and 15 wt %, between 15 wt % and 30 wt %, etc.

In one embodiment, the poloxamer used in the composition is Pluronic® F-127. In Pluronic® F-127, the sum of two a's in the above block polymer structure may be 200, and b may have a value of 65. In Pluronic® F-127, the ratio of the sum of two a's to b in the poloxamer (i.e., a:b) may be from 2:1 to 4:1. Tables 1 and 2 illustrate chemical composition and specifications of Pluronic® F-127.

TABLE 1
Chemical Composition of Pluronic ® F-127

| | | | | | | Number | | |
|---|---|---|---|---|---|---|---|---|
| | | | | | | average | | |
| | | | | Content of | | molecular | Poly- | Number of |
| | | | | Oxyethylene | Molecular | weight | dispersity | repeating |
| Pluronic | Poloxamer | a | b | (Percent) | Weight | (g/mol) | index | units |
| F 127 NF | 407 | 101 | 56 | 71.5-74.9 | 9840-14600 | 10000 | 1.2 | EG₂₀₀PG₆₅PH₀ |

TABLE 2
Specifications of Pluronic ® F-127

| | pH | Cloud | | | | | Ethylene | Propylene | 1,4 |
|---|---|---|---|---|---|---|---|---|---|
| Physical | (2.5% | point, | APHA | | BHT, | Unsaturation | Oxide, | Oxide, | dioxane, |
| Form | in water) | 10% | color | % H₂O | ppm | mEq/g | ppm | ppm | ppm |
| Solid | 5.0-7.5 | >100° C. | 120 | Cast sold 0.4 max. | 50-125 | 0.048 a 0.017 | 1 max. | 5 max. | 0.002% |
| | | | max. | Prill 0.75 max. | | | | | max. |

### Stabilization Polymer

In addition, in one set of embodiments, the composition may comprise xanthan gum, and/or another stabilization polymer. Examples of other stabilization polymers include hyaluronic acid, alginic acid, modified celluloses such as hydroxypropyl methylcellulose, or others such as described herein. Xanthan gum generally refers to a high molecular weight polysaccharide used as a food additive and rheology modifier, as would be known by those of ordinary skill in the art. In addition, many such xanthan gums are readily available commercially. Xanthan gum may be produced, as a non-limiting example, by a process involving fermentation of glucose or sucrose by the Xanthonmonas campestris bacterium. In some embodiments, the backbone of the polysaccharide chain may have two beta-D-glucose units linked through the 1 and 4 positions. The side chains are formed of two mannose and one glucuronic acid, so the chain has repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. Two of these chains may be aligned to form a double helix, giving a rather rigid rod configuration that accounts for its high efficiency as a viscosifier of water.

However, it should be understood that not all xanthan gums have precisely the above molecular configuration or properties, and that xanthan gums may vary in molecular composition, e.g., depending on the source of the xanthan gum, especially those arising from different biological sources. In addition, other stabilization polymers instead of (or in addition to) xanthan gum can be used, for example, KELTROL® BT and/or KELTROL® RD, KELZAN® XC, KELZAN® XCD, KELZAN® D, KELZAN® CC, XANTURAL® 180, XANTURAL® 75, or the like, all of which can be obtained commercially from various suppliers.

The molecular weight of the xanthan gum or other stabilization polymer can vary. For instance, the xanthan gum or other stabilization polymer may have any suitable molecular weight, for example, at least about 1 million, at least about 2 million, at least about 5 million, at least about 10 million, at least about 25 million, or at least about 50 million. In other embodiments, the molecular weight can vary from about one million to 50 million, e.g., depending upon various factors such as how it is prepared. In some embodiments, the molecular weight can range from approximately 1 million to approximately 25 million, e.g., as measured by a Brookfield Viscometer or other suitable device. In yet other embodiments, the molecular weight may be, for example, 1, 2, 3, 4, or 5 (+/−0.5) million, or 6, 7, 8, 9, 10, 11, 12, 13, 14, 15, 16, 17, 18, 19, 19, 20, 21, 22, 23, 24, or 25 (+/−2) million. Still other molecular weights are also possible.

The xanthan gum (and/or another stabilization polymer) may be present within the composition at concentrations of at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, the xanthan gum and/or other stabilization polymer may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the xanthan gum and/or other stabilization polymer may be present at between 0.1 wt % and 10 wt %, between 1 wt % and 5 wt %, between 0.5 wt % and 2 wt %, between 0.5 wt % and 5 wt %, between 0.5 wt % and 2 wt %, or the like.

### Active Ingredient—Melatonin

In one set of embodiments, the composition may also comprise an active ingredient. The active ingredient may be one suitable for treating PCOS and/or endometriosis. The active ingredient may be one suitable for treating PCOS. The active ingredient may be one suitable for treating endometriosis. For instance, the active ingredient may be one suitable for treatment of a subject for a condition when the active ingredient is delivered to the vagina, or another suitable body cavity. In some embodiments, the active ingredient is present in a therapeutically effective amount. One or more than one active ingredient may be used, depending on the embodiment.

For example, in certain embodiments, the active ingredient may be one that is suitable for treating PCOS and/or for treating endometriosis. In some embodiments, a composition such as is described herein may be inserted into the vagina of a subject, e.g., to or treating PCOS and/or for treating endometriosis. In one embodiment, for example, the composition may contain melatonin and/or a salt thereof as an active ingredient.

Melatonin (chemical name: N-[2-(5-methoxy-1H-indol-3-yl)ethyl]acetamide) has been known since the early 1950s (U.S. Pat. No. 2,546,658), has the following chemical structure:

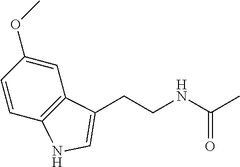 [SMILES: COc1ccc2[nH]cc(CCNC(C)=O)c2c1]

Melatonin is a hormone produced primarily by the pineal gland in the brain and was known initially for its relationship with sleep. The bioactivity of melatonin is related to numerous behavioral, endocrinological, and immune processes. These activities can be mediated by receptor dependent or independent mechanisms. Melatonin is relatively small and has an amphiphilic nature that makes it very lipid soluble, so melatonin can easily pass through tissues and reach all of the cell compartments. While melatonin is well known for its sleep inducing qualities, it has also been shown to be a potent antioxidant. The antioxidant activity of melatonin is mediated in several ways. Melatonin has direct anti-oxidant capacity by scavenging reactive oxygen species and nitrogen-based reactants. It has also been shown to induce additional antioxidants, such as superoxide dismutase, glutathione peroxidase, glutathione reductase and catalase. Melatonin has also been shown to induce the production of glutathione and is able to maintain a high reduced glutathione to oxidized glutathione ratio (GSH/GSSG) (Slominski).

As used herein, melatonin disclosed herein is understood to include all enantiomers, diastereomers, racemates and mixtures thereof. The drug can be a salt, a solvate, a hydrate, a pro-drug, a co-crystal, a derivative, in free base form, or a mixture thereof.

Possible dose ranges of melatonin are from 1 mg/day to 1000 mg/day, or from 1 mg/day to 900 mg/day or from 1 mg/day to 800 mg/day, or from 1 mg/day to 700 mg/day, or from 1 mg/day to 600 mg/day, or from 1 mg/day to 500 mg/day. In some embodiments, dose ranges are from 1 mg/day to 400 mg/day, or from 1 mg/day to 300 mg/day, or from 1 mg/day to 200 mg/day, or from 1 mg/day to 100 mg/day. In some embodiments, the dose range is from 1 mg/day to 90 mg/day, or from 1 mg/day to 80 mg/day, or from 1 mg/day to 70 mg/day, or from 1 mg/day to 60 mg/day, or from 1 mg/day to 50 mg/day, or from 1 mg/day to 40 mg/day, or from 1 mg/day to 30 mg/day, or from 1 mg/day to 20 mg/day, or from 1 mg/day or from 10 mg/day, or from 1 mg/day to 5 mg/day. In some embodiments, the dose ranges of melatonin are from 10 mg/day to 1000 mg/day, or from 10 mg/day to 900 mg/day, or from 10 mg/day to 800 mg/day, or from 10 mg/day to 800 mg/day, or from 10 mg/day to 700 mg/day, or from 10 mg/day to 600 mg/day, or from 10 mg/day to 500 mg/day, or from 10 mg/day to 400 mg/day, or from 10 mg/day to 300 mg/day, or from 10 mg/day to 200 mg/day, or from 10 mg/day to 100 mg/day, or from 10 mg/day to 50 mg/day.

In embodiments, the dose is in a range that provides a plasma or serum concentration of melatonin of less than about 1000 mg/mL, or less than about 900 mg/mL, or less than about 800 mg/mL, or less than about 700 mg/mL, or less than about 600 mg/mL, or less than about 500 mg/mL, or less than about 400 mg/mL, or less than about 200 mg/mL, or less than about 100 mg/mL, or less than about 50 mg/mL, or less than about 40 mg/mL, or less than about 30 mg/mL, or less than about 20 mg/mL, or less than about 10 mg/mL, or less than about 5 mg/mL, or less than about 1 mg/mL, or less than about 0.5 mg/mL, or less than about 0.1 mg/mL, or less than about 0.05 mg/mL, or less than about 0.01 mg/mL.

In some embodiments, the does is in a range that provides a plasma or serum concentration of melatonin in the range from 0.01 mg/mL to 1000 mg/mL, or from 1 mg/mL to 1000 mg/mL, or from 10 mg/mL to 1000 mg/mL, or from 50 mg/mL to 1000 mg/mL, or from 100 mg/mL to 1000 mg/mL, or from 200 mg/mL to 1000 mg/mL, or from 300 mg/mL to 1000 mg/mL, or from 400 mg/mL to 1000 mg/mL, or from 500 mg/mL to 1000 mg/mL, or from 600 mg/mL to 1000 mg/mL, or from 700 mg/mL to 1000 mg/mL, or from 800 mg/mL to 1000 mg/mL, or from 900 mg/mL to 1000 mg/mL.

### Active Ingredient—Melatonin Receptor Agonist

In one set of embodiments, the composition may also comprise an active ingredient. The active ingredient may be one suitable for treating PCOS and/or endometriosis. The active ingredient may be one suitable for treating PCOS. The active ingredient may be one suitable for treating endometriosis. For instance, the active ingredient may be one suitable for treatment of a subject for a condition when the active ingredient is delivered to the vagina, or another suitable body cavity. In some embodiments, the active ingredient is present in a therapeutically effective amount. One or more than one active ingredient may be used, depending on the embodiment.

For example, in certain embodiments, the active ingredient may be one that is suitable for treating PCOS and/or for treating endometriosis. In some embodiments, a composition such as is described herein may be inserted into the vagina of a subject, e.g., to or treating PCOS and/or for treating endometriosis. In one embodiment, for example, the composition may contain melatonin receptor agonist and/or a salt thereof as an active ingredient. Melatonin receptor agonists are analogues of melatonin that bind to and activate the melatonin receptor.

In some embodiments, the melatonin receptor agonist includes ramelteon (ROZEREM), agomelatine (VALDOXAN, MELITOR, THYMANAX) or tasimelteon (HETLIOZ).

As used herein, a melatonin receptor agonist includes one or more of N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, agomelatine, CGP 52608, low-dose melatonin A, GR196429, S20242, S23478, S24268, S25150, melatonin receptor research compound A, GW290569, controlled release melatonin, luzindole, GR135531, melatonin agonist A, melatonin analogue B, melatonin agonist C, melatonin agonist D, melatonin agonist E, melatonin agonist F, melatonin agonist G, melatonin agonist H, melatonin agonist I, melatonin analog J, melatonin analog K, melatonin analog L, AH-001, GG-012, enol-3-IPA, ML-23, SL-18.1616, IP-100-9, melatonin low-dose B, sleep inducing peptide A, oros-melatonin, AH-017, AH-002, IP-101, 5-hydroxy-N-acetyl-tryptamine (NAT), 5-methoxy-N-bu-tanoyltryptamine (bMT), prazosin, phenylmelatonin, seradrene, β-methyl-6-chloromelatonin, 5-hydroxyethoxy-N-acetyltryptamine (5-HEAT), 8-methoxy-2-propionamidotetralin, PD-6735, seroctin, N-[2-(5-methoxy-2-phenylfuro[2,3-b]pyridin-3-yl)ethyl]acetamide, N-[2-(5-methoxy-2-phenylfuro[2,3-c]pyridin-3-yl)ethyl]acetamide, N-[(±)-2-(7-methoxy-1,2,3,4-tetrahydro-1-naphthyl)ethyl]cyclopropyl-carboxamide, N-[2-(7-methoxy-1-naphthyl)ethyl]acetamide, N-acetyl-4-aminomethyl-6-methoxy-9-methyl-1,2,3,4-tetrahydrocarbazole (AMMTC), 3-(2-aminopropyl)indole, 6-chloromelatonin, 2,3-dihydromelatonin, 6-chloro-2,3-dihydromelatonin, N-acetyl-N′-formyl-5-methoxykynurenamine, 6-methoxybenzoxazolinone, or a pharmaceutically acceptable salt, solvate, clathrate, polymorph, or co-crystal thereof.

Possible dose ranges of a melatonin receptor agonist are from 1 mg/day to 1000 mg/day, or from 1 mg/day to 900 mg/day or from 1 mg/day to 800 mg/day, or from 1 mg/day to 700 mg/day, or from 1 mg/day to 600 mg/day, or from 1 mg/day to 500 mg/day. In some embodiments, dose ranges are from 1 mg/day to 400 mg/day, or from 1 mg/day to 300 mg/day, or from 1 mg/day to 200 mg/day, or from 1 mg/day to 100 mg/day. In some embodiments, the dose range is from 1 mg/day to 90 mg/day, or from 1 mg/day to 80 mg/day, or from 1 mg/day to 70 mg/day, or from 1 mg/day to 60 mg/day, or from 1 mg/day to 50 mg/day, or from 1 mg/day to 40 mg/day, or from 1 mg/day to 30 mg/day, or from 1 mg/day to 20 mg/day, or from 1 mg/day or from 10 mg/day, or from 1 mg/day to 5 mg/day. In some embodiments, the dose ranges of melatonin are from 10 mg/day to 1000 mg/day, or from 10 mg/day to 900 mg/day, or from 10 mg/day to 800 mg/day, or from 10 mg/day to 800 mg/day, or from 10 mg/day to 700 mg/day, or from 10 mg/day to 600 mg/day, or from 10 mg/day to 500 mg/day, or from 10 mg/day to 400 mg/day, or from 10 mg/day to 300 mg/day, or from 10 mg/day to 200 mg/day, or from 10 mg/day to 100 mg/day, or from 10 mg/day to 50 mg/day.

In embodiments, the dose is in a range that provides a plasma or serum concentration of a melatonin receptor agonist of less than about 1000 mg/mL, or less than about 900 mg/mL, or less than about 800 mg/mL, or less than about 700 mg/mL, or less than about 600 mg/mL, or less than about 500 mg/mL, or less than about 400 mg/mL, or less than about 200 mg/mL, or less than about 100 mg/mL, or less than about 50 mg/mL, or less than about 40 mg/mL, or less than about 30 mg/mL, or less than about 20 mg/mL, or less than about 10 mg/mL, or less than about 5 mg/mL, or less than about 1 mg/mL, or less than about 0.5 mg/mL, or less than about 0.1 mg/mL, or less than about 0.05 mg/mL, or less than about 0.01 mg/mL.

In some embodiments, the does is in a range that provides a plasma or serum concentration of a melatonin receptor agonist in the range from 0.01 mg/mL to 1000 mg/mL, or from 1 mg/mL to 1000 mg/mL, or from 10 mg/mL to 1000 mg/mL, or from 50 mg/mL to 1000 mg/mL, or from 100 mg/mL to 1000 mg/mL, or from 200 mg/mL to 1000 mg/mL, or from 300 mg/mL to 1000 mg/mL, or from 400 mg/mL to 1000 mg/mL, or from 500 mg/mL to 1000 mg/mL, or from 600 mg/mL to 1000 mg/mL, or from 700 mg/mL to 1000 mg/mL, or from 800 mg/mL to 1000 mg/mL, or from 900 mg/mL to 1000 mg/mL.

### Additional Active Ingredient(s)

The pharmaceutical formulation may also include one or more additional active ingredient(s). The pharmaceutical formulation may also include an antibiotic as an active ingredient in certain cases. In another set of embodiments, the active ingredient may be an ingredient that can be delivered to the vagina, e.g., for local or systemic delivery to the subject. In some embodiments, the active ingredient may be any active ingredient that can be dissolved and/or suspended within a composition as described herein, for example, a gel. For example, the active ingredient may be one that is at least partially water soluble. The gel may be relatively viscous, e.g., as discussed herein. Non-limiting examples of suitable active ingredients include, but are not limited to, lidocaine, hydrocortisone, progesterone, misoprostol, metronidazole, ketoconazole, clobetasol, acyclovir, miconazole, nonoxynol-9, lactobacilli, tinidazole, butoconazole, flucytosine, glycerol monolaurate (GML), or the like.

Possible classes of additional active pharmaceutical ingredients include anticholinergic agents, analgesics, anesthetics, antiarthritics, anti-inflammatory agents, antiasthma drugs, urinary tract disinfectants, anticoagulants, anticonvulsants, antidepressants, antidiabetics, antineoplastics, antipsychotics, antihypertensives, muscle relaxants, antiprotozoals, spermicidals, antibacterials, antihistamines and decongestants, antiparasitic compounds, antiviral compounds, steroidal hormones, opiates, SERMs, SARMs, SPRMs, aromatase inhibitors. In embodiments, drug classes that can be used include steroidal hormones, opiates, antimuscarinics, SERMs, SARMs, SPRMs and aromatase inhibitors. In embodiments, the additional pharmaceutical ingredient is progesterone, estrogen, estradiol, tamoxifen, raloxifen, lasofoxifene, ospemifen, enobosarm, ulipristal, ulipristal acetate, anastrazole, oxybutynin, tolterodine, trospium, solifenacin, darifenacin, dicyclomine, propantheline, propiverine, bethanecol, methylbenactyzium and scopolamine, particularly oxybutynin and progesterone. In embodiments, the one or more active pharmaceutical ingredients includes an anticholinergic agent, such as tolterodine, trospium, solifenacin, darifenacin, dicyclomine, propantheline, propiverine, bethanecol, methylbenactyzium and scopolamine. In embodiments, the anticholinergic agent is an antimuscarinic agent, particularly oxybutynin.

### Salts

If a salt is present, the salt may be a pharmaceutically acceptable salt in some embodiments. Pharmaceutically acceptable salts include salts which are, within the scope of sound medical judgment, suitable for use in contact with the tissues of a subject (e.g., a human) without undue toxicity, irritation, allergic response, and the like, and are commensurate with a reasonable benefit/risk ratio. Pharmaceutically acceptable salts may also be salts that are generally safe, non-toxic and neither biologically nor otherwise undesirable and includes that which is acceptable for human pharmaceutical use. Pharmaceutically acceptable salts are well known in the art. Pharmaceutically acceptable salts of the compounds describe herein include those derived from suitable inorganic and organic acids and bases. Examples of pharmaceutically acceptable, nontoxic acid salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N′(Cl₄alkyl)₄ salts. Representative alkali or alkaline earth metal salts include sodium, lithium, potassium, calcium, magnesium, and the like. Further pharmaceutically acceptable salts include, when appropriate, quaternary salts.

Release of Active Ingredient from Composition

In some cases, the active ingredient may be released from the composition over any suitable period of time. For example, a therapeutically effective amount of the active ingredient may be released from 1 to 100 days. In some instances, a therapeutically effective amount of the active ingredient may be released up to about 1 to 30 days, up to about 1 to 28 days, up to about 1 to 21 days, up to about 1 to 7 days, about 5 or 15 days, or about 8 to 29 days following administration. As still other examples, the active ingredient may be released at therapeutically effective amounts from the composition, following application to the subject, for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days. In some embodiments, only a single application of the composition to the subject may be needed. However, in other embodiments, the composition may be applied to the subject more than once, for example, in applications separated by any of the lengths of time discussed herein.

For example, in some cases, tₘₐₓ, or the time at which the maximum concentration of the active ingredient is present in the vagina (due to release by the composition) may be at least 1 day, at least 2 days, at least 3 days, etc., or other times such as those described above. In addition, in some embodiments, tₘₐₓ may be less than 30, 29, 28, 27, 26, 25, 24, 23, 22, 21, 20, 19, 18, 17, 16, 15, 14, 13, 12, 11, 10, 9, 8, 7, 6, 5, 4, 3, or 2 days. Combinations of any of these are also possible, e.g., tₘₐₓ may be between 1 and 3 days, or between 4 and 6 days, etc. Without wishing to be bound by any theory, it is believed that this may be due to the relatively high viscosity of the composition and/or slower release kinetics from the composition.

The active ingredient may be present within the composition at any suitable concentration. For example, the active ingredient may be present at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc.

In addition, in some cases, the active ingredient may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the active ingredient may be present at between 1 wt % and 5 wt %, between 0.5 wt % and 2 wt %, between 0.5 wt % and 5 wt %, or the like.

In addition, in accordance with certain embodiments, the composition may release the active ingredient over an extended period of time. In some cases, this may be determining by determining a concentration of an active ingredient in the mucus of the vagina after a certain period of time, for instance, after 1 day, 2 days, 3 days, etc. For example, in one set of embodiments, the concentration of the active ingredient may be at least 100 micrograms per gram composition (micrograms/g), at least 200 micrograms/g, at least 300 micrograms/g, at least 400 micrograms/g, at least 500 micrograms/g, at least 600 micrograms/g, at least 700 micrograms/g, etc. The mucus may be sampled, for example, using a swab, or other techniques known to those of ordinary skill in the art.

In addition, in accordance with certain embodiments, the composition may release the active ingredient over an extended period of time. In some cases, this may be determining by determining a concentration of an active ingredient in the mucus of the vagina after a certain period of time, for instance, after 1 day, 2 days, 3 days, etc. For example, in one set of embodiments, the concentration of the active ingredient may be at least 100 micrograms/g, at least 200 micrograms/g, at least 300 micrograms/g, at least 400 micrograms/g, at least 500 micrograms/g, at least 600 micrograms/g, at least 700 micrograms/g, etc. The mucus may be sampled, for example, using a swab, or other techniques known to those of ordinary skill in the art.

Other components may be present as well within a composition. As a non-limiting example, in one set of embodiments, the composition may include citrate and/or a citrate salt. These may include, for example, citric acid, citric acid monohydrate, sodium citrate, sodium citrate dihydrate, or the like. Other examples include other suitable salts, e.g., to make citrate buffer such as sodium phosphate, potassium phosphate, or the like. Buffers such as these may be used, for example, to maintain the pH of the composition (for example, at around 4.5, or another suitable pH). As another example, the composition may include benzyl alcohol. Benzyl alcohol may be useful, for example, as a solvent or a preservative.

Components such as these may each independently be present in any suitable amount or concentration. For example, a component may be present at a concentration of at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, a component may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the component may be present at between 1 wt % and 5 wt %, between 0.5 wt % and 2 wt %, between 0.5 wt % and 5 wt %, between 0.5 wt % and 2 wt %, or the like.

In addition, in one set of embodiments, water may be present within the composition. Any suitable amount of water may be present, for example, such that the composition forms a gel, has a relatively high viscosity as discussed herein, or the like. For example, in some cases, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the composition may be water.

In one set of embodiments, the composition is a gel. The can may be semi-solid material that includes a relatively large amount or concentration of water, e.g., as noted above. In some cases, the polymer (e.g., one or more poloxamers) may from a scaffold structure that contains the water within the gel.

The gel or other composition, e.g., as described herein, may have a relatively high viscosity, at least in one set of embodiments. Those of ordinary skill in the art will be aware of techniques for determining viscosity of a sample, for example, using devices such as a rheometers, viscometers, etc.

In some cases, the composition may have a viscosity at room temperature of at least 1 million cP, at least 1.1 million cP, at least 1.2 million cP, at least 1.3 million cP, at least 1.4 million cP, at least 1.5 million, at least 1.6 million cP, at least 1.8 million cP, at least 2 million cP, at least 2.2 million cP, at least 2.4 million cP, at least 2.6 million cP, at least 2.8 million cP, at least 3 million cP, at least 3.5 million cP, at least 4 million cP, etc. In addition, in certain embodiments, the composition may have a viscosity of no more than 4 million cP, no more than 3.5 million cP, no more than 3 million cP, no more than 2.8 million cP, no more than 2.6 million cP, no more than 2.4 million cP, no more than 2.2 million cP, no more than 2.0 million cP, no more than 1.8 million cP, no more than 1.6 million cP, no more than 1.5 million cP, no more than 1.4 million cP, no more than 1.3 million cP, no more than 1.2 million cP, no more than 1.1 million cP, no more than 1.0 million cP, etc. Combinations of any of these are also possible, for example, the composition may exhibit a viscosity of between 1.5 million cP and 2 million cP, between 1.8 million cP and 2.4 million cP, between 1.2 million cP and 3 million cP, etc.

In some cases, the composition may contain xanthan gum and/or another stabilization polymer, and a polymer such as a poloxamer, which may cause the composition to have a relatively high viscosity. In some embodiments, the composition may contain no other component that changes the viscosity of said composition at room temperature by more than +/−100,000 centipoise.

Due to the natural acidity of the vagina, vaginal compositions typically aim for a pH of about 4.5. In embodiments, the pH of the composition may be between about 4 and about 7. In embodiments, the pH of the composition may be between about 4 and about 5.5. In embodiments, the pH of the composition may be between about 4 and about 5. In embodiments, the pH of the composition may be between about 4.5 and about 5.5. For example, the composition may have pH about 4, 4.1, 4.2, 4.3, 4.4, 4.5, 4.6, 4.7, 4.8, or 4.9. In embodiments, vaginal compositions as described herein (e.g., using melatonin) have up to about pH 5.5, for example pH 5.0, 5.1, 5.2, 5.3, 5.4, or 5.5. In embodiments, vaginal compositions as described herein (e.g., using melatonin) have up to about pH 7, for example pH 5.6, 5.7, 5.8, 5.9, 6.0, 6.1, 6.2, 6.3, 6.4, 6.5, 6.6, 6.7, 6.8, 6.9, or 7.0.

In embodiments, a composition as described herein may release about 50% of the active ingredient (e.g., melatonin or a melatonin receptor agonist) within the composition in about 1 hour to about 80 hours. In embodiments, a composition as described herein may release about 50% of the active ingredient (e.g., melatonin or a melatonin receptor agonist) within the composition in about 10 hours to about 70 hours. In embodiments, a composition as described herein may release about 50% of the active ingredient (e.g., melatonin or a melatonin receptor agonist) within the composition in about 20 hours to about 70 hours. In embodiments, a composition as described herein may release about 50% of the active ingredient (e.g., melatonin or a melatonin receptor agonist) within the composition in about 10 hours to about 60 hours. In embodiments, a composition as described herein may release about 50% of the active ingredient (e.g., melatonin or a melatonin receptor agonist) within the composition in about 10 hours to about 50 hours. In embodiments, a composition as described herein may release about 50% of the active ingredient (e.g., melatonin or a melatonin receptor agonist) within the composition in about 10 hours to about 40 hours. In embodiments, a composition as described herein may release about 50% of the active ingredient (e.g., melatonin or a melatonin receptor agonist) within the composition in about 10 hours to about 30 hours. In embodiments, a composition as described herein may release about 50% of the active ingredient (e.g., melatonin or a melatonin receptor agonist) within the composition in about 15 hours to about 60 hours. In embodiments, a composition as described herein may release about 50% of the active ingredient (e.g., melatonin or a melatonin receptor agonist) within the composition in about 15 hours to about 50 hours. In embodiments, a composition as described herein may release about 50% of the active ingredient (e.g., melatonin or a melatonin receptor agonist) within the composition in about 15 hours to about 40 hours. In embodiments, a composition as described herein may release about 50% of the active ingredient (e.g., melatonin or a melatonin receptor agonist) within the composition in about 15 hours to about 30 hours. In embodiments, a composition as described herein may release about 50% of the active ingredient (e.g., melatonin or a melatonin receptor agonist) within the composition in about 20 hours to about 60 hours. In embodiments, a composition as described herein may release about 50% of the active ingredient (e.g., melatonin or a melatonin receptor agonist) within the composition in about 20 hours to about 50 hours. In embodiments, a composition as described herein may release about 50% of the active ingredient (e.g., melatonin or a melatonin receptor agonist) within the composition in about 20 hours to about 40 hours. In embodiments, a composition as described herein may release about 50% of the active ingredient (e.g., melatonin or a melatonin receptor agonist) within the composition in about 20 hours to about 30 hours. The time may be any value or subrange within the recited ranges.

In embodiments, a composition as described herein may release about 80% of the active ingredient (e.g., melatonin or a melatonin receptor agonist) within the composition in about 1 hour to about 80 hours. In embodiments, a composition as described herein may release about 80% of the active ingredient (e.g., melatonin or a melatonin receptor agonist) within the composition in about 10 hours to about 70 hours. In embodiments, a composition as described herein may release about 80% of the active ingredient (e.g., melatonin or a melatonin receptor agonist) within the composition in about 20 hours to about 70 hours. In embodiments, a composition as described herein may release about 80% of the active ingredient (e.g., melatonin or a melatonin receptor agonist) within the composition in about 10 hours to about 60 hours. In embodiments, a composition as described herein may release about 80% of the active ingredient (e.g., melatonin or a melatonin receptor agonist) within the composition in about 10 hours to about 50 hours. In embodiments, a composition as described herein may release about 80% of the active ingredient (e.g., melatonin or a melatonin receptor agonist) within the composition in about 10 hours to about 40 hours. In embodiments, a composition as described herein may release about 80% of the active ingredient (e.g., melatonin or a melatonin receptor agonist) within the composition in about 10 hours to about 30 hours. In embodiments, a composition as described herein may release about 80% of the active ingredient (e.g., melatonin or a melatonin receptor agonist) within the composition in about 15 hours to about 60 hours. In embodiments, a composition as described herein may release about 80% of the active ingredient (e.g., melatonin or a melatonin receptor agonist) within the composition in about 15 hours to about 50 hours. In embodiments, a composition as described herein may release about 80% of the active ingredient (e.g., melatonin or a melatonin receptor agonist) within the composition in about 15 hours to about 40 hours. In embodiments, a composition as described herein may release about 80% of the active ingredient (e.g., melatonin or a melatonin receptor agonist) within the composition in about 15 hours to about 30 hours. In embodiments, a composition as described herein may release about 80% of the active ingredient (e.g., melatonin or a melatonin receptor agonist) within the composition in about 20 hours to about 60 hours. In embodiments, a composition as described herein may release about 80% of the active ingredient (e.g., melatonin or a melatonin receptor agonist) within the composition in about 20 hours to about 50 hours. In embodiments, a composition as described herein may release about 80% of the active ingredient (e.g., melatonin or a melatonin receptor agonist) within the composition in about 30 hours to about 60 hours. In embodiments, a composition as described herein may release about 80% of the active ingredient (e.g., melatonin or a melatonin receptor agonist) within the composition in about 40 hours to about 50 hours. The time may be any value or subrange within the recited ranges.

In embodiments, a composition as described herein may release about 100% of the active ingredient (e.g., melatonin or a melatonin receptor agonist) within the composition in about 1 hour to about 80 hours. In embodiments, a composition as described herein may release about 100% of the active ingredient (e.g., melatonin or a melatonin receptor agonist) within the composition in about 10 hours to about 70 hours. In embodiments, a composition as described herein may release about 100% of the active ingredient (e.g., melatonin or a melatonin receptor agonist) within the composition in about 20 hours to about 70 hours. In embodiments, a composition as described herein may release about 100% of the active ingredient (e.g., melatonin or a melatonin receptor agonist) within the composition in about 10 hours to about 60 hours. In embodiments, a composition as described herein may release about 100% of the active ingredient (e.g., melatonin or a melatonin receptor agonist) within the composition in about 10 hours to about 50 hours. In embodiments, a composition as described herein may release about 100% of the active ingredient (e.g., melatonin or a melatonin receptor agonist) within the composition in about 10 hours to about 40 hours. In embodiments, a composition as described herein may release about 100% of the active ingredient (e.g., melatonin or a melatonin receptor agonist) within the composition in about 10 hours to about 30 hours. In embodiments, a composition as described herein may release about 100% of the active ingredient (e.g., melatonin or a melatonin receptor agonist) within the composition in about 15 hours to about 60 hours. In embodiments, a composition as described herein may release about 100% of the active ingredient (e.g., melatonin) within the composition in about 20 hours to about 60 hours. In embodiments, a composition as described herein may release about 100% of the active ingredient (e.g., melatonin or a melatonin receptor agonist) within the composition in about 30 hours to about 60 hours. In embodiments, a composition as described herein may release about 100% of the active ingredient (e.g., melatonin or a melatonin receptor agonist) within the composition in about 40 hours to about 60 hours. In embodiments, a composition as described herein may release about 100% of the active ingredient (e.g., melatonin or a melatonin receptor agonist) within the composition in about 45 hours to about 60 hours. The time may be any value or subrange within the recited ranges.

In addition, in one set of embodiments, the composition may have a relatively low concentration or amount of air. For instance, in one embodiment, the composition is substantially free of air. In some cases, during manufacture, a large amount of air may be introduced into the composition, e.g., as foam or bubbles, etc. However, this air may be undesirable in accordance with certain embodiments, and accordingly, the composition may be prepared by also including a step of removing air from the composition that has been introduced during manufacture.

In some embodiments, the composition, after removing at least some of the air may contain no more than 20 vol %, no more than 15 vol %, no more than 12 vol %, no more than 10 vol %, no more than 8 vol %, no more than 6 vol %, no more than 5 vol %, no more than 4 vol %, no more than 3 vol %, no more than 2 vol %, or no more than 1 vol %, etc. of air. In some cases, the air may be removed such that no air bubbles are visually present within the composition.

Without wishing to be bound by any theory, it is believed that the presence of air may reduce the viscosity of the composition, e.g., making it easier for the composition to flow. Accordingly, in some embodiments, any air that is introduced may be removed, thereby increasing the viscosity of the composition, e.g., to at least 1 million cP, or other ranges of viscosities, such as any of those described herein. Furthermore, in some cases, removing the air may also increase the density of the final composition. For instance, the density of the composition may be at least 0.98 g/cm³, at least 0.99 g/cm³, at least 1 g/cm³, at least 1.01 g/cm³, at least 1.02 g/cm³, at least 1.03 g/cm³, at least 1.05 g/cm³, at least 1.1 g/cm³, etc. In some cases, the density of the composition may be no more than 1.1 g/cm³, no more than 1.05 g/cm³, no more than 1.03 g/cm³, no more than 1.02 g/cm³, no more than 1.01 g/cm³, etc. Furthermore, in some embodiments, combinations of any of these ranges are possible. For example, the final density of the composition may be between 1.00 g/cm³ and 1.01 g/cm³, between 0.99 g/cm³ and 1.02 g/cm³, etc.

In one embodiment, air may be removed from a composition, e.g., during or after formation, by applying a pressure less than atmospheric or ambient pressure to the composition. For instance, the pressure that is applied may be less than 1 bar, less than 800 mbar, less than 600 mbar, less than 500 mbar, less than 400 mbar, less than 300 mbar, less than 200 mbar, less than 100 mbar, less than 75 mbar, less than 60 mbar, less than 50 mbar, less than 40 mbar, less than 30 mbar, less than 20 mbar, less than 10 mbar, less than 5 mbar, less than 3 mbar, less than 2 mbar, less than 1 mbar, etc. It should be noted that 1 atmosphere is approximately 1 bar, and that these pressures are absolute pressures (i.e., a pressure of less than about 1 bar means a pressure lower than atmospheric pressure, i.e., a vacuum pressure). Such pressures may be applied for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc. In addition, in some cases, the pressure may be applied until the composition comprises less than a certain amount of air, e.g., less than 15 vol %, or other percentages such as those described herein. As yet another example, in some cases, a solution may be caused to form a gel by removing a certain amount of air from the solution.

In certain embodiments, as another example, the air may be removed from the composition using a Versator or other deaerator, degasser, and/or defoamer. In a Versator, a material such as a liquid is spread onto the inside of a rotating Versator disc under vacuum to remove entrapped air, foam, gas, etc. While the liquid travels across the disc, the high vacuum draws off the bubbles, etc., from the liquid. Versators may be obtained from several commercial sources. Accordingly, in some embodiments, a composition such as described herein may be treated using a Versator for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein

As yet another example, in some cases, the air may be removed from the composition using centrifugation. Without wishing to be bound by any theory, it is believed that by centrifuging the composition, air (being less dense) may be forced out of the composition. Thus, for example, the material may be centrifuged at any suitable speed, e.g., at least at least 500 RPM, at least 1,000 RPM, at least 2,000 RPM, at least 3,000 RPM, at least 5,000, at least 10,000 RPM, or the like, for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein.

Furthermore, it should be understood that these techniques are non-limiting, and that other methods of removing air, besides centrifuges or Versators, are also possible in still other embodiments.

In embodiments, the product can be manually or filled using an automated filling machine. In some embodiments, the automated filling machine (e.g., a Capmatic automated filing machine) is especially designed to fill applicator-based products, such as vaginal applicator-based products. As the viscosity of the product decreased at refrigerated temperatures, e.g., at temperatures of about 4° C. (e.g., a reduction in viscosity of 2-3×), the product may become suitable for automated filling into a vaginal applicator. In some embodiments, a cooled, jacketed vessel, feeding cooled product into the filler hopper to fill the applicators is used.

### Implantable Delivery Device

In some embodiments, the active ingredient is administered via an implantable drug delivery device. Non-limiting examples can be found in U.S. Pat. Nos. 7,833,545; 7,838,024; 7,883,718; 7,829,112; 8,057,817; 8,062,658; 8,506,988; 8,404,272; 8,992,968; 9,370,574; 8,399,013; 9,084,717; 9,308,168; and PCT Pub. Nos. WO2015/153817; WO2018/215772.

In embodiments, the device comprises: an inner core comprising one or more active pharmaceutical ingredients distributed throughout a first water-insoluble polymer; an intermediate coating positioned around the inner core, said intermediate coating comprising an acrylate polymer; and an outer coating positioned around the intermediate coating, said outer coating comprising a second water-insoluble polymer; wherein the acrylate polymer is formed from one or more monomers of formula:

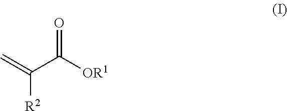

wherein R1 is selected from H, alkyl, alkenyl, alkynyl, or aryl; and R2 is selected from H or alkyl. It has been found that an implantable drug delivery device including the above features provides a reduction in the initial drug burst, resulting in improved controlled release. The implantable drug delivery device disclosed herein delivers the active agent (e.g., melatonin or a melatonin receptor agonist) continuously, with generally linear release kinetics, in a controlled manner and can be used for treating medical conditions for extended periods of time, such as several days, weeks or months as desired. Without wishing to be bound by theory, it is thought that the water-insoluble polymer of the outer coating (i.e. the second water-insoluble polymer) acts as a protective barrier that, over the duration of use, prevents moisture ingress without impeding permeation of the drug, allowing the acrylate polymer of the intermediate coating to control the rate of release of the drug from the inner core. A further benefit associated with this reduction in initial drug burst is that a lower drug loading is possible, which results in material cost savings and reduced drug wastage.

The acrylate polymer of the intermediate coating provides additional benefits as it adheres well to the inner core, such that it may be easily applied, allowing for straight-forward manufacture of the implantable device. This is in contrast to silicone-based polymer coatings, which are cured onto the inner core at a high temperature—this can lead to poor adhesion and/or damage to the materials of the inner core.

The drug delivery device disclosed herein also has suitable mechanical properties e.g. good flexibility, allowing for convenient implantation.

In a second aspect, the present invention provides melatonin or a melatonin receptor agonist for use in treating PCOS or for treating endometriosis, wherein the melatonin or a melatonin receptor agonist is delivered via the device according to any embodiments described herein.

As used herein, the one or more active pharmaceutical ingredients (e.g., melatonin or a melatonin receptor agonist) comprised by the device disclosed herein are understood to include all enantiomers, diastereomers, racemates and mixtures thereof. The drug can be a salt, a solvate, a hydrate, a pro-drug, a co-crystal, a derivative, in free base form, or a mixture thereof.

As used herein, the term “polymer” takes its usual definition the art and so refers to a homopolymer or copolymer formed from the polymerisation of one or more monomers.

As used herein, the term “homopolymer” takes its usual definition in the art, and so refers to a polymer whose polymer chains comprise one type of monomer. As used herein, the term “co-polymer” takes its usual definition in the art, and so refers to a polymer whose polymer chains comprise two or more different types of monomers.

As used herein, the term “monomer” takes its usual definition in the art and so refers to a molecular compound that may chemically bind to another monomer to form a polymer. As used herein, the term “water-insoluble polymer” refers to polymers wherein less than 10 mg of the polymer dissolves in 100 ml water at 25° C. This definition applies to both the first water-insoluble polymer (i.e. the water-insoluble polymer of the inner core) and the second water-insoluble polymer (i.e. the water-insoluble polymer of the outer coating) of the implantable drug delivery device.

As used herein, an “implantable drug delivery device” takes its usual definition in the art, and refers to a drug delivery device that is implanted in a bodily orifice or in bodily tissue. Implantable drug delivery devices that are implanted in a bodily orifice include intravaginal implants, rectal implants, and urethral implants. Implantable drug delivery devices that are implanted in bodily tissue include subcutaneous implants, intravascular implants, intraocular implants, intrathecal implants, peritoneal implants, intravesical implants, and intrauterine implants.

Preferably, the implantable drug delivery device is an intravaginal implant or a subcutaneous implant. These implantable drug delivery devices typically remain in-situ for a more prolonged period (e.g. for over 28 days) in comparison to other implantable devices. The drug delivery system disclosed herein is capable of providing controlled release over extended periods of time (e.g. over 28 days). As such the drug delivery system disclosed herein is particularly well-suited for an intravaginal implant or a subcutaneous implant. More preferably, the implantable drug delivery device is an intravaginal ring. Intravaginal rings are adapted for delivery of an active agent to the cervical region. The mechanical properties of the drug delivery system mean it is particularly well-suited to intravaginal rings.

The inner core of the implantable drug delivery device disclosed herein comprises one or more active pharmaceutical ingredients distributed throughout a first water-insoluble polymer. The first water-insoluble polymer, together with the one or more pharmaceutical ingredients, forms the majority of the inner core. The first water-insoluble polymer, together with the one or more pharmaceutical ingredients, can also form the entirety of the inner core.

As used herein, the term “distributed throughout a first water-insoluble polymer” means that the one or more active pharmaceutical ingredient (e.g., melatonin or a melatonin receptor agonist) is dissolved or dispersed homogenously throughout the first water-insoluble polymer. This relationship between the one or more active pharmaceutical ingredients and the water-insoluble polymer of the inner core (i.e. the first water-insoluble polymer) may be thought of as a drug-polymer matrix. By encapsulating the one or more active pharmaceutical ingredients within a polymer matrix, the drug may be released as desired from the inner core during normal use, but in the event of the device fracturing, any leakage of the drug(s) from the core is minimized.

The reduced drug burst and improved controlled release is not dependent on fractionating the inner core of the implantable device into segments. However, the device is still compatible with having a segmented inner core. When the inner core is segmented, the inner core comprises multiple segments, each segment of the inner core comprising a water-insoluble polymer, which may be same or different with respect to the water-insoluble polymer of neighbouring segments, and the one or more active pharmaceutical ingredients are distributed throughout the water-insoluble polymer of at least one of the segments. Where there are multiple active pharmaceutical ingredients present in this context, these may be located in the same or in different segments.

Preferably, the inner core is not fractionated into multiple segments, as this further simplifies the manufacturing process of the device. Therefore, the implantable drug delivery device preferably comprises a unitary inner core comprising one or more active pharmaceutical ingredients distributed throughout a first water-insoluble polymer. As used herein, the term “unitary” when used in the context of the inner core refers to the core being of a single segment.

Suitable water-insoluble polymers for the inner core include modified polyolefins (such as ethylene vinyl acetate) polyurethanes, cross-linked silicones, polyesters, polyamides, polyethers, cellulose, and water-insoluble derivatives of cellulose. Preferably, the water-insoluble polymer of the inner core is ethylene vinyl acetate with 10 to 90 wt % of vinyl acetate, based on the total weight of the ethylene vinyl acetate polymer. Using an ethylene vinyl acetate polymer with 10 to 90 wt % of vinyl acetate provides good homogenous distribution of active pharmaceutical ingredient throughout the water-insoluble polymer of the inner core. Preferably, the water-insoluble polymer of the inner core is ethylene vinyl acetate with 15-45 wt % of vinyl acetate, based on the total weight of the ethylene vinyl acetate polymer. In a particularly preferred embodiment, the water-insoluble polymer of the inner core is an ethylene vinyl acetate polymer with 28 wt % vinyl acetate, based on the total weight of the ethylene vinyl acetate polymer. This gives particularly good results, as can be seen from the examples.

The implantable drug delivery device disclosed herein further comprises an intermediate coating positioned around the inner core. The intermediate coating completely coats the inner core irrespective of the shape of the inner core. Said intermediate coating comprises an acrylate polymer formed from one or more monomers of formula (I):

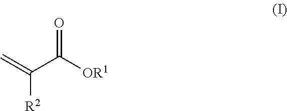

wherein R1 is selected from H, alkyl, alkenyl, alkynyl, or aryl; and R2 is selected from H or alkyl.

The term “acrylate polymer” when used in its general sense in the art refers to a homopolymer or copolymer formed from one or more monomers that comprise a vinyl group directly bonded to a carboxylic acid/carboxylic ester group. In the context of the present disclosure, the specific acrylate polymers used in the intermediate coating disclosed herein are formed from one or more monomers of formula (I). Preferably, the acrylate polymer of the intermediate coating is a co-polymer.

As used herein, the term “alkyl” refers to C1-C10 alkyl group, which can be linear or branched. Preferably, alkyl is C1-C4 alkyl, such as methyl, ethyl, n-propyl or t-butyl, more preferably methyl or ethyl.

As used herein, the term “alkenyl” refers to C2-C10 alkenyl group. Preferably, it is a C2-C4 alkenyl group. Examples include vinyl, allyl, 1-propenyl, isopropenyl and 1-butenyl.

As used herein, the term “alkynyl” refers to a C2-C10 alkynyl group which can be linear or branched. Preferably, it is a C2-C4 alkynyl group. As used herein, the term “aryl” refers to a monocyclic, bicyclic, or tricyclic aromatic group containing from 1 to 14 carbon atoms in the aromatic system, such as phenyl, biphenyl, naphthyl, anthracenyl, preferably phenyl.

Preferably, R1 is selected from alkyl, alkenyl, alkynyl, or aryl (where these terms can take any of the above meanings). More preferably R1 is alkyl, particularly C1-C4 alkyl, more preferably methyl or ethyl. In a particularly preferred embodiment, the acrylate polymer is a co-polymer, and the one or more monomers comprise a first monomer wherein R1 is ethyl, and a second monomer wherein R1 is methyl.

Preferably, R2 is selected from H or C1-C4 alkyl, more preferably wherein R2 is selected from H or methyl. In a particularly preferred embodiment, the acrylate polymer is a co-polymer, and the one or more monomers comprise a first monomer wherein R2 is H, and a second monomer wherein R2 is alkyl, preferably C1-C4 alkyl, more preferably methyl.

In a particularly preferred embodiment, the acrylate polymer of the intermediate coating is a co-polymer of ethyl acrylate and methyl methacrylate. As the skilled person will appreciate, these monomers have the following structures:

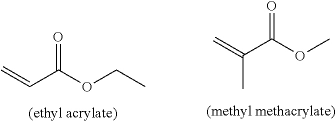

As can be seen from the above structures, in the instance that the acrylate polymer is a co-polymer of ethyl acrylate and methyl methacrylate, the one or more monomers of formula (I) comprise, or consist of, a first monomer wherein R1 is ethyl and R2 is H (ethyl acrylate) and a second monomer wherein R1 is methyl and R2 is methyl (methyl methacrylate).

Most preferably, the acrylate polymer of the intermediate coating is poly(ethyl acrylate, methyl methacrylate), where the ratio of ethyl acrylate:methyl methacrylate is 2:1. Such acrylate polymers are available under trade names Eudragit NE 30D or Eudragit NM 30 D. These acrylate polymers provide particularly good reduction in the burst effect and an improved controlled release profile.

The acrylate polymer forms the majority of the intermediate coating. The acrylate polymer can also form the entirety of the intermediate coating. Preferably, the amount of acrylate polymer in the intermediate coating is at least 80 wt %, more preferably, at least 90 wt %, based on the total weight of the intermediate coating. In addition to the acrylate polymer, the intermediate coating may additionally comprise at least one further component, such as plasticisers, fillers and/or pore forming agents. These additional components are optional, but may impart further benefits. Plasticisers provide flexibility and reduce the possibility and degree of cracking of the acrylate polymer. Plasticisers such as polyethylene glycol, triacetin, diethyl phthalate, dibutyl phthalate, tributyl citrate, triethyl citrate are preferred, as they further improve flexibility without having a detrimental effect on the release profile of the drug. Polyethylene glycol plasticisers are particularly preferred. Fillers, such as talc, provide increased structural rigidity and may be used to enhance the cosmetic appearance of the product.

The implantable drug delivery device disclosed herein further comprises an outer coating positioned around the intermediate coating, said outer coating comprising a second water-insoluble polymer. The outer coating completely coats the intermediate coating irrespective of the shape of the intermediate coating. The second water-insoluble polymer forms the majority of the outer coating. The second water-insoluble polymer can also form the entirety of the outer coating.

Suitable water-insoluble polymers for the outer coating include polyurethanes, ethylene vinyl acetate with 10 to 90 wt % of vinyl acetate based on the total weight of the ethylene vinyl acetate polymer, and cross-linked silicone polymers. Preferably, the water-insoluble polymer of the outer coating is an ethylene vinyl acetate polymer with 10 to 90 wt % of vinyl acetate, more preferably 20 to 60 wt % of vinyl acetate, based on the total weight of the ethylene vinyl acetate polymer. In a particularly preferred embodiment, the water-insoluble polymer of the outer coating is an ethylene vinyl acetate polymer with 40 wt % of vinyl acetate, based on the total weight of the ethylene vinyl acetate polymer. This gives particularly good results, as can be seen from the examples.

The implantable drug delivery device may further comprise one or more additional layers between the inner core and the intermediate coating, and/or between the intermediate coating and the outer coating. Additional layers may provide certain properties to the device, for example cosmetic enhancement, improved handling of the device, improved mechanical properties and/or improved compression properties. Alternatively, the intermediate coating may be in direct contact with the inner core, and/or the outer coating may be in direct contact with the intermediate coating.

The minimum and maximum thickness of the intermediate coating may be tailored to achieve a particular release rate for the specific drug in question. In terms of the minimum thickness, the thickness of the intermediate coating may be at least 1 μm, at least 10 μm, at least 50 μm, or at least 75 μm. Preferably, the thickness of the intermediate coating is at least 50 μm. In terms of the maximum thickness, the thickness of the intermediate coating may be less than 1000 μm, less than 500 μm, less than 250 μm, or less than 150 μm. Preferably, the thickness of the intermediate coating is less than 100 μm.

The minimum and maximum thickness of the outer coating may be tailored depending on the level of protection that the intermediate coating requires from the in-vivo environment. In terms of the minimum thickness, the thickness of the outer coating may be at least 10 μm, at least 25 μm or at least 50 m. Preferably, the thickness of the outer coating is at least 10 m. In terms of the maximum thickness, the thickness of the outer coating is less than 2000 m, less than 1000 μm, less than 500 μm or less than 250 m. Preferably, the thickness of the outer coating is less than 50 μm.

The implantable drug delivery device may be used to deliver a variety of one or more active pharmaceutical ingredients for treatment of PCOS and/or endometriosis. In embodiments the pharmaceutical ingredient includes melatonin or a melatonin receptor agonist.

In other embodiments, an additional pharmaceutical ingredient is also included. The implantable drug delivery device is particularly well-suited to delivering one or more active pharmaceutical ingredients with a narrow therapeutic window. As used herein a drug with a “narrow therapeutic window” is a drug where there is a small dosage range between the minimum therapeutic dose and the dose where unwanted side effects are observed. It is particularly important in this context to minimize the initial drug burst and achieve controlled release.

The implantable drug delivery device may be used to treat a variety of different medical conditions such PCOS and endometriosis.

The implantable drug delivery device is particularly well-suited to treating medical conditions where localized delivery and controlled release are of particular importance. The preferable form of the implantable drug delivery device in this context is as an intravaginal ring. The skilled person will appreciate that an “intravaginal ring” is a ring shaped device, suitable for placement in the vaginal tract.

In embodiments, the melatonin or a melatonin receptor agonist is delivered via the device disclosed herein. The total amount of the one or more pharmaceutical ingredients present in the inner core may be tailored depending on the therapeutic requirements in question. The total amount of the one or more pharmaceutical ingredients in the inner core is at least 2 wt %, at least 5 wt %, at least 10 wt %, or at least 20 wt %, based on the total weight of the inner core. In embodiments, the total amount of the one or more pharmaceutical ingredients in the inner core is at least 5 wt % based on the total weight of the inner core. In terms of the maximum amount, the total amount of the one or more pharmaceutical ingredients in the inner core is less than 50 wt %, less than 40 wt %, or less than 30 wt % based on the total weight of the inner core. In embodiments, the total amount of the one or more pharmaceutical ingredients in the inner core is less than 30 wt % based on the total weight of the inner core.

The therapeutically effective dose of the drug that is locally delivered by the device disclosed herein can be tailored depending on the drug in question.

Preferably the device delivers the desired dose of active agent for a period of at least 7 days. More preferably, the device delivers the desired dose of drug for a period of at least 28 days.

The implantable drug delivery device may take a variety of different shapes. The skilled person will be familiar with suitable shapes that are appropriate for the specific implantable delivery device in question.

The implantable drug delivery device may be manufactured according to a variety of different methods familiar to the skilled person. The inner core may be conveniently manufactured by any conventional moulding technique, for example, by hot melt extrusion of the active agent and core polymer, by injection moulding of the device or by 3-D printing. The intermediate coating may be applied by any coating technique, for example, coextrusion, overmoulding, spray film coating, dip coating and the like. A preferred method involves spray coating of a latex emulsion comprising the intermediate coating onto the inner core or dip coating of the inner core in a dispersion or solution of the intermediate coating. The outer coating may be applied by the same coating methods as those that can be used for applying the intermediate coating, e.g. over-moulding the prefabricated section, or, by way of lamination by hot pressing the device between two layers of the outer coating of the required thickness.

### Segmented Ring (Ethylene-Vinyl-Acetate (EVA), Intravaginal Ring (IVR)

In some embodiments, the active ingredient (e.g., melatonin or a melatonin receptor agonist) is administered via a segmented, e.g., an ethylene-vinyl-acetate (EVA), intravaginal ring (IVR). In some embodiments, EVA-based segmented intravaginal rings (IVRs) are designed to release melatonin or melatonin receptor agonist. In an aspect of the disclosure, the disclosed EVA rings are used to treat PCOS in a subject. In another aspect, the disclosed EVA rings are used to treat endometriosis in a subject.

Disclosed herein are segmented IVRs that deliver a relatively constant dose of melatonin or melatonin receptor agonist. The IVRs described herein are composed of EVA, and are formulated as matrix devices. The IVRs disclosed herein can be used to treat PCOS and/or endometriosis.

The segmented rings described herein are advantageous as compared to other EVA rings systems in that the loading of segments can be optimized within a narrow range of EVA concentrations from about 27% to about 36%. This range, in about 28% vinyl acetate content ensures a stable system that avoids being at or around a transition point between fully dispersed drug in polymer and crystalline drug particles dispersed in polymer. The second advantage of the EVA ring systems described herein is the ability to easily change release rates through variations in segment length. Thus, preparation of clinical supplies for dose ranging studies is straight forward.

In some aspects of the invention described herein, where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. For example, a range from 27% to 36% would include 27% to 29%, or 27% to 33% or 33% to 35%, etc. Such ranges would also include individual points in the range, for example, 28%, 29%, 30% etc.

The disclosed IVRs herein are designed to have a rate of release of melatonin or melatonin receptor agonist from about 1 mg/day to about 1000 mg/day.

Exemplary IVRs are disclosed in U.S. Patent Pub. Nos. 2021/0007976 and 2021/0000641; each of which is incorporated herein by reference in its entirety. Such IVRs may be made in a manner similar to that described previously (Kimball A B, Javorsky E, Ron E S, Crowley W, Langer R. A novel approach to administration of peptides in women: Systemic absorption of a GnRH agonist via transvaginal ring delivery system. Journal of Controlled Release. 2016; 233:19-28; see also U.S. Patent Pub. Nos. 2021/0007976 and 2021/0000641; each of which is incorporated herein by reference in its entirety).

In some embodiments, a segment of the IVRs disclosed herein are in the range of about 74.5 mm to about 156.0 mm. In some embodiments the segment is in the range of about 74.5 mm to about 148.5 mm. In some embodiments the segment of the IVRs disclosed herein is about 74.5 mm, about 148.5 mm or about 156 mm.

In some embodiments, the IVRs are about 57 mm in diameter with a cross-section diameter of about 5 mm.

### Methods of Use

Accordingly, as discussed above, a variety of compositions are covered in various embodiments, including any suitable device or composition for administration of melatonin or a melatonin receptor agonist, e.g. for intravaginal administration.

In embodiments, combinations of any of the above-described components, such as poloxamer, xanthan gum and/or another stabilization polymer, an active ingredient, and water may be used. For example, in one set of embodiments, at least 50 wt %, at least 60 wt %, at least 70 wt %, at least 80 wt %, at least 90 wt %, at least 95 wt %, or at least 99 wt % of the composition comprises or consists essentially of polymer such as a poloxamer, xanthan gum and/or another stabilization polymer, an active ingredient, and water. The active ingredient includes, for example, melatonin or a melatonin receptor agonist as well as salts thereof.

In embodiments, an implantable device, such as an intravaginal device, may be used.

Furthermore, as mentioned, certain aspects as described herein are generally directed to compositions and methods for applying such compositions and devices for the treatment or prevention of indications such as any of those described herein, e.g., to the vagina of a subject, such as a human. The subject can also be a non-human animal. In embodiments, the subject is female.

In one set of embodiments, a composition or device such as is described herein may be used to treat PCOS and/or to treat endometriosis. In some cases, a subject may be treated by applying a composition or device such as is described herein to the vagina to treat PCOS and/or to treat endometriosis. The composition may have a suitable active ingredient, such as melatonin or a melatonin receptor agonist, and/or a salt thereof. After insertion, the active ingredient may be released, e.g., into the vagina or cervix, which may thus treat PCOS and/or to treat endometriosis.

### Polycystic Ovarian Syndrome (PCOS)

Polycystic ovarian syndrome (PCOS) is a disease occurring in females, and is characterized by hyperandrogenism. PCOS represents a spectrum of phenotypes associated with follicle growth arrest, chronic anovulation, minimal granulosa cell proliferation, hyperthecosis, hyperandrogenemia, central adiposity and insulin resistance. In PCOS, plasma luteinizing hormone (LH) is high and FSH is low, increasing the LH/FSH ratio, typically due to increased LH or GnRH (Gonadotropin releasing hormone) pulse. In PCOS, disturbances occur in ovarian steroid synthesis, hyperinsulinemia being one of the condition which increases ovarian androgen production and stimulate LH and increases LH/GnRH secretion.

There are no approved treatments for PCOS. Optimal first line treatment of PCOS in adolescents remains controversial. Lifestyle changes (dietary and exercise modification) followed by either oral contraceptive pills (OCP) to control symptoms of hyperandrogenism is normally recommended, which has not more than 50% clinical success. However, there is significant variability in clinical practice, depending on whether the physician and patient's primary goal of treatment is to treat the symptoms of hyperandrogenism or the features of metabolic syndrome. Additionally, in clinical practice anti-androgenic medications such as spironolactone, flutamide, and insulin sensitizing agents such as metformin or pioglitazone are used as add-on therapy when OCP or metformin fail to produce the clinically desired outcomes, yet their use in adolescent population is not recommended. Anti-androgens like spironolactone are second choice having risk of electrolyte imbalance. GnRH analogue is the next line of therapy to reduce androgen with lack of potency needing additional hormonal therapy to counteract the chemical menopause that they induce. Metformin may be administered to reduce androgen level, improve menstrual regularity and fertility as well as insulin resistance. Clomiphene with or without metformin improves conception rates with risk of ovarian hyperstimulation and multiple pregnancies.

### Endometriosis

Endometriosis is one of the most common gynecological diseases in the United States. It is a painful, often debilitating disease that affects more than 6.5 million women in America between the ages of 15 and 4. Endometriosis occurs when the lining of the uterus grows ectopically, most commonly on the ovaries, fallopian tubes, tissues that hold the uterus in place, and the outer surface of the uterus. While less common, endometrial growths can also be found on the vagina, cervix, vulva, bowel, bladder, or rectum. The most common symptoms of endometriosis include pain, bleeding or spotting, infertility, and digestive problems such as diarrhea, constipation, bloating, or nausea. As used herein, “endometrium” refers to the mucous membrane lining the uterus. The endometrium changes throughout the menstrual cycle. Menstruation is the cyclic, sloughing of the endometrium in response to hormonal fluctuations. The menstrual cycle is divided into two phases, the follicular or proliferative phase, and the luteal or secretory phase. The proliferative phase is characterized by the development of ovarian follicles. The secretory phase is typically 14 days long and begins after ovulation.

As used herein, “endometrial cells” refer to cells from the endometrium. Endometrial cells can be subdivided into stromal cells and epithelial cells.

As used herein, “stromal cells” refer to connective tissue cells of any organs. Stromal cells support the function of the parenchymal cells of that organ. Endometrial stromal cells are important in the initiation and maintenance of pregnancy.

As used herein, “epithelial cells” refer to cells that form cohesive sheets of cells referred to as epithelia. They function as a covering or lining for body surfaces, and as functional units of secretory glands. Epithelial cells can be specialized for absorption, secretion, or to act as a barrier.

As used herein, “endometriosis” refers to a gynecological disease wherein tissue from the uterus grows in the abdominal cavity, outside of the uterus. The two main symptoms of endometriosis include pain and infertility. The main causes of the pain and infertility are endometrial implants and adhesions. As used herein, “endometrial implants” refer to endometrial tissue found in ectopic locations. Implants resemble small, flat patches on the peritoneal surface of the pelvic region. These implants cause irritation and inflammation in the surrounding tissue, which can lead to the formation of adhesions. “Adhesions” as used herein refer to bands of internal scar tissue that can bind tissues and organs that are normally mobile.

Endometriosis is classified in “stages” based on the severity of the disease, the extent of spread of the disease, the involvement of pelvic structures, the extent of pelvic adhesions, and blockage of the fallopian tubes. Staging of endometriosis does not necessarily reflect the severity of symptoms experienced by the patient. The four stages of endometriosis are equivalent to minimal, mild, moderate, and severe. The majority of patients fall within minimal and mild stages. Minimal endometriosis, called Stage I, is characterized by isolated implants and no significant adhesions. Mild endometriosis, Stage II, is characterized by superficial implants less than 5 cm in aggregate without significant adhesions. Stage I and Stage II endometriosis are often combined in the same category called “superficial endometriosis”. Moderate (Stage III) endometriosis is characterized by the appearance of endometriomas, which are a type of cyst that forms when endometrial tissue grows in the ovaries. Severe endometriosis (Stage IV) is characterized by multiple implants, cysts, and severe adhesions which lead to scarring around the tubes and ovaries. Women with stage IV endometriosis are the most likely to have infertility problems.

### Dosing

In embodiments, the composition is administered to a subject between one and ten times. In embodiments, the composition is administered to a subject between one and ten times per menstrual cycle. In embodiments, the composition is administered to a subject between one and five times per menstrual cycle. In embodiments, the composition is administered to a subject between one and three times per menstrual cycle. In embodiments, the composition is administered to a subject one time per menstrual cycle. In embodiments, the composition is administered to a subject twice per menstrual cycle.

In embodiments, the composition is administered daily. In embodiments, the composition is administered every two days. In embodiments, the composition is administered every 3, 4, 5, 6, 7, 8, 9, 10, 20, 28, or 30 days. In embodiments, the composition is administered as needed, for example when the subject is in pain.

Terms such as “treat,” “treatment,” “treating,” etc. comprise therapeutic treatment of subjects having already developed a disease, in particular in manifest form. Therapeutic treatment may be symptomatic treatment in order to relieve the signs and/or symptoms of the disease or causal treatment in order to reverse, partially reverse, stop, or slow down the progression of the disease. Thus, the compositions and methods of the present disclosure may be used, for instance, as therapeutic treatment (e.g., for acute or chronic therapy).

Additionally, terms such as “prevent,” “preventing,” or “prevention” generally refer to the reduction of the occurrence of the disease, and/or a sign and/or symptom thereof, in the treated sample relative to an untreated control sample, or delays the onset of one or more signs and/or symptoms of the disease relative to the untreated control sample, in a statistically significant manner. Preventing the disease, and/or a sign and/or a symptom thereof, includes preventing or delaying the initiation of the disease, sign, and/or symptom. Prevention also includes preventing a recurrence of the disease, sign, and/or symptom.

In certain aspects, the composition can be applied to a subject, e.g., to the vagina of a subject, and/or to another body cavity, for example, the mouth or the rectum. Any suitable technique may be used to apply the composition to the subject. For instance, the composition may be free or mass flowing, e.g., so that it may be administered through an applicator or other suitable device. Thus, in some embodiments, the composition may be contained within applicator, such as a vaginal applicator or a syringe, which can be applied, e.g., by the subject, or by another person.

The subject may be, but is not limited to, humans (i.e., of any age group, e.g., a pediatric subject (e.g., infant, child, adolescent) or adult subject (e.g., young adult, middle-aged adult, or senior adult)) and/or other non-human animals, for example, mammals (e.g., primates (e.g., monkeys such as cynomolgus monkeys or rhesus monkeys, chimpanzees, etc.); commercially relevant mammals such as cattle, pigs, horses, sheep, rabbits, mice, rats, goats, cats, dogs, etc.) and birds (e.g., commercially relevant birds such as chickens, ducks, geese, turkeys, etc.). In certain embodiments, the subject is a mammal. The subject may have a uterus. The subject may have a vagina. In embodiments, the subject is a human with a vagina and/or a uterus. In embodiments, the subject is pre-menopausal.

In one set of embodiments, as discussed, the composition is applied to treat the subject with a therapeutically effective amount of an active ingredient (e.g., melatonin). The therapeutically effective amount may be an amount which, when administered to a subject for treating or preventing a disease, is sufficient to effect such treatment or prevention PCOS and/or endometriosis.

### Kits

Still another aspect of the present disclosure is directed to kits. The kit may include a package or an assembly including one or more of the compositions as described herein, and/or other compositions. Each of the compositions of the kit may be provided in liquid form (e.g., in solution), or in solid form (e.g., a dried powder), or in gaseous form in some cases. In certain cases, some of the compositions may be constitutable or otherwise processable (e.g., to an active form), for example, by the addition of a suitable solvent or other species, which may or may not be provided with the kit. Examples of other components include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

In an embodiment, the kit includes one or more implantable delivery devices as described herein.

A kit may, in some cases, include instructions in any form that are provided in connection with the compositions described herein in such a manner that one of ordinary skill in the art would recognize that the instructions are to be associated with those compositions. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In an embodiments, provided herein are kits comprising the compositions and gels described herein. For example, the kit includes an applicator suitable for vaginal application. In other examples, the applicator is pre-filled with the compositions or the gels described herein. In other embodiments, the applicator is not-prefilled with the compositions or the gels described herein. In some embodiments, the compositions or gels described herein are at refrigerated temperatures (e.g., about 4° C.) prior to pre-filling the applicator. In some embodiments, the kit includes an applicator that is pre-filled with the composition, where the composition includes a poloxamer, a stabilization polymer, and an active ingredient (e.g., melatonin or a melatonin receptor agonist for treating PCOS and/or treating endometriosis.

In some embodiments, the kit further includes one or more instructions for inserting the applicator into the vagina. In other embodiments, the kit includes instructions for treating PCOS and/or treating endometriosis by applying the applicator filled with the compositions or gels described herein. In some embodiments, the kit further includes instructions for filling the applicator with the composition or gel.

In some embodiments, the compositions described herein are stored at room temperature (either pre-filled in an applicator or a separate storage container). In some embodiments, the compositions can be stored for 1 day, 2 days, 10 days, 2 weeks, 1 month, 2 months, 3 months, 4 months, 6 months, 7 months, 8 months, 9 months, 10 months, 11 months, 1 year, or more at room temperature.

### EXAMPLES

The following examples are intended to illustrate certain embodiments of the present disclosure, but do not exemplify the full scope of the disclosure.

### Example 1: Composition

In this prophetic example, a composition in accordance with one embodiment is as follows (all components USP grade). The active ingredient may be melatonin or a melatonin receptor agonist.

TABLE 1
composition

| Material | g/100 g |
|---|---|
| Poloxamer 407 | 16.50 |
| Xanthan gum | 2.00 |
| Active ingredient | 2.00 |
| Citric acid monohydrate | 0.60 |
| Sodium citrate dihydrate | 0.55 |
| Benzyl alcohol | 1.00 |
| Purified water (qs) | 77.35 |
| Total | 100 |

### Example 2: Method of Making the Composition

This prophetic example illustrates a method of making the composition described in Example 1, in accordance with another embodiment.

Purified water is added to a kettle (or other suitable container), which is placed under a dissolver (30-60 HP) with a 12-inch stand dissolver blade. The dissolver is started, and an active ingredient is added to the kettle. The dissolver is allowed to mix for at least 10 minutes, or until the active ingredient has visually dissolved in the water.

Afterwards, mixing is continued while citric acid monohydrate and sodium citrate dihydrate are added to the kettle. The kettle is then cooled with chilled cooling water, e.g., a temperature of 8° C. Mixing is continued for at least 5 minutes, or until these have visually dissolved.

Mixing and cooling then continues while benzyl alcohol and poloxamer 407 is added. These are mixed until the water is clear and everything has visually dissolved.

Mixing and cooling continues while xanthan gum is slowly added. Mixing of the xanthan gum continues for at least 10 minutes, or until the composition is visually uniform. The speed of mixing may be adjusted, for example, as the composition thickens and its viscosity increases, and/or to avoid trapping too many air bubbles within the composition.

Next, the composition is transferred into a round-bottom, jacketed, stainless steel pressure/vacuum kettle, or another suitable container. The kettle is cooled as before, e.g., using chilled cooling water at 8° C. Mixing in the kettle is started and the batch is slowly recirculated under a vacuum. Mixing, cooling with chilled cooling water, and recirculating under vacuum occurs for at least 30 minutes, or until the composition has been sufficiently deaerated.

It should be understood that the above is by way of example only, and that other techniques for making the compositions described herein are also contemplated. For example, the addition sequence of poloxamer 407 and xanthan gum may be reversed, with the poloxamer going into the mixing first. Air may be removed using a centrifuge, a Versator, or the like.

### Example 3: Method of Making the Composition

This prophetic example illustrates a method of making a composition in accordance with another embodiment.

The primary compounding phase in this example is prepared by adding 154 kg of Purified Water USP to a 270 L kettle. The kettle is placed under a dissolver equipped with a 10-inch standard blade. Mixing commences at about 600 rpm. 4.86 kg of an active ingredient (melatonin or a melatonin receptor agonist) is added and mixed for 10 minutes until visually dissolved. Mixing continues and 1200 g citric acid and 1100 g of sodium citrate are added and mixed for 6 min until visually dissolved. Cooling of the batch is started during this step by flowing chilled water through the kettle jacket.

Mixing continues and 2000 g of benzyl alcohol and 33.0 kg of poloxamer 407 are added. Cooling and mixing continue until the solution is clear and the poloxamer is visually dissolved. The batch temperature reaches approximately 11° C. during this step. This step creates foam on the surface of the batch and the mixing speed is decreased to allow the foam to dissipate. Mixing speed is then increased, eventually reaching 1200 rpm for addition of the 4.00 kg of xanthan gum. After the addition of the xanthan gum, the product rapidly increases in viscosity and the original mixing blade size is switched to a 12-inch blade to better facilitate mixing at this stage of the process. A 14-inch blade may also be used at this scale.

After this step in the process, the product generates a large amount of foaming. The foaming can be reduced by transferring the product into a cooling, jacketed vacuum vessel and recirculate the product for several passes to help remove the air. Another approach is to use a Versator, which is a device for removing air from liquids and semi-solids. The product was passed through the Versator several times until all air was removed. Measurements of the viscosity after Versator treatment rose to over 3 million cP, from 1.2 million cP at room temperature prior to using the Versator.

### Example 4: Air Removal

In this example, a manufacturing process is outlined describing air removal from the composition, as well as filing the composition into vaginal applicators is provided.

During mixing, 2000 g of benzyl alcohol and 33.0 kg of poloxamer 407 is added. Cooling and mixing continued until the solution is clear and the poloxamer is visually dissolved. The batch temperature reached approximately 11° C. during this step. This step creates foam on the surface of the batch and the mixing speed is decreased to allow the foam to dissipate. Mixing speed is then increased, eventually reaching 1200 rpm for addition of the 4.00 kg of xanthan gum. After the addition of the xanthan gum, the product rapidly builds viscosity. A switch may be made to a 12-inch blade to better facilitate mixing at this stage of the process.

After this step in the process, the product generates a large amount of foaming. Initially, the product is transferred into a cooling, jacketed vacuum vessel and recirculated the product for several passes to help remove the air. A second approach used a Versator, which is a device especially designed to remove air from liquids and semi-solids. The product was passed through the Versator several times until all air was removed. Measurements of the viscosity after Versator treatment rise from 1.2 Cp (prior to use of the Versator) to over 3M Cp at room temperature.

Following this step, the product is manually or filled using an automated filling machine. In some embodiments, the automated filling machine (e.g., a Capmatic automated filing machine) is especially designed to fill vaginal applicator-based products. As the viscosity of the product decreased at refrigerated temperatures, e.g., at temperatures of about 4° C. (e.g., a reduction in viscosity of 2-3×) provides a product suitable for automated filling into a vaginal applicator. In some embodiments, a cooled, jacketed vessel, feeding cooled product into the filler hopper to fill the applicators is used.

### Example 5: Implantable Delivery Device: 10% w/w Melatonin or a Melatonin Receptor Agonist—EVA (28% VA Content) Core

A 10% w/w blend of melatonin or a melatonin receptor agonist base and EVA (ethyl vinyl acetate) (28% VA—vinyl acetate) content) is prepared and hot melt extruded at 110° C., to produce a solid solution, and pelletized. The pellets are injection molded using a barrel temperature of 95° C. and a tool temperature of 35° C. to form a ring.

In-vitro dissolution is performed on the intravaginal ring of above in 200 ml acetate buffer pH 4.1 at 37° C., and 100 rpm over a 28 day period using shaking incubator. Dissolution samples are typically taken after 1, 2, 3, 4, 7, 14, 21, 28 days. Dissolution samples are analyzed by HPLC.

### Definitions

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.” The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

When ranges are given by specifying the lower end of a range separately from the upper end of the range, it will be understood that the range can be defined by selectively combining any one of the lower end variables with any one of the upper end variables that is mathematically possible. Where ranges are recited, it will be understood that any subrange or value within the recited ranges, including endpoints, is contemplated.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition, comprising:
a poloxamer;
a stabilization polymer; and
an active ingredient for treating polycystic ovarian syndrome (PCOS) and/or treating endometriosis,
wherein the composition has a viscosity at room temperature of at least 1.5 million cP.

2. The composition of claim 1, where the composition is a gel.

3. The composition of any one of claim 1 or 2, wherein the composition has a viscosity at room temperature of at least 2 million cP.

4. The composition of any one of claims 1-3, wherein the composition has a viscosity at room temperature of at least 3 million cP.

5. The composition of claim 1, wherein the composition contains no other component that changes the viscosity of said composition at room temperature by more than +/−100,000 centipoise.

6.-21. (canceled)

22. The composition of claim 1, wherein the active ingredient comprises melatonin, a melatonin receptor agonist, and/or a pharmaceutically acceptable salt thereof.

23. The composition of claim 22, wherein the active ingredient comprises melatonin or a melatonin receptor agonist.

24. The composition of claim 1, wherein the active ingredient is present at 1-5 wt %.

25.-36. (canceled)

37. A method, comprising:
applying, to a vagina of a subject, a composition comprising a poloxamer, a stabilization polymer, and an active ingredient for treating polycystic ovarian syndrome (PCOS) and/or treating endometriosis, wherein the composition, as applied, has a viscosity of at least 1.5 million cP.

38. The method of claim 37, wherein the active ingredient comprises melatonin, a melatonin receptor agonist, and/or a pharmaceutically acceptable salt thereof.

39.-52. (canceled)

53. A composition, comprising:
a poloxamer;
a stabilization polymer; and
an active ingredient for treating polycystic ovarian syndrome (PCOS) and/or treating endometriosis, wherein the composition is made by a process comprising forming a composition comprising the poloxamer, the stabilization polymer, and the active ingredient, and removing air from the composition.

54. The composition of claim 53, wherein the active ingredient comprises melatonin, a melatonin receptor agonist, and/or a pharmaceutically acceptable salt thereof.

55. (canceled)

56. The composition of claim 53, wherein the composition has a viscosity at room temperature of at least 1.5 million cP.

57.-92. (canceled)

93. A kit comprising a poloxamer: a stabilization polymer; and an active ingredient for treating polycystic ovarian syndrome (PCOS) and/or treating endometriosis,
wherein the composition has a viscosity at room temperature of at least 1.5 million cP, wherein the kit includes an applicator suitable for vaginal application.

94.-100. (canceled)

101. An implantable drug delivery device comprising:
an inner core comprising at least one active pharmaceutical ingredient distributed throughout a first water-insoluble polymer, wherein the at least one active pharmaceutical ingredient comprises melatonin or a melatonin receptor agonist or pharmaceutically acceptable salt thereof; an intermediate coating positioned around the inner core, said intermediate coating comprising an acrylate polymer; and an outer coating positioned around the intermediate coating, said outer coating comprising a second water-insoluble polymer;
wherein the acrylate polymer is formed from one or more monomers of formula (I): 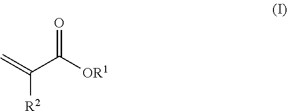
wherein R¹ is selected from H, alkyl, alkenyl, alkynyl, or aryl; and R² is selected from H or alkyl.

102. The device according to claim 101 wherein the first water-insoluble polymer is an ethylene vinyl acetate polymer with 10-90 wt % of vinyl acetate, based on the total weight of the ethylene vinyl acetate polymer.

103.-117. (canceled)

118. The device of according to claim 101, wherein the device comprising melatonin or a melatonin receptor agonist treats PCOS and/or treats endometriosis.

119.-129. (canceled)

130. An ethylene-vinyl-acetate (EVA), intravaginal ring (IVR), wherein the ring contains at least two segments/fibers, wherein at least one segment contains melatonin or a melatonin receptor agonist.

131. The EVA ring according to claim 130, wherein the EVA ring releases about 1 mg/day to about 1000 mg/day melatonin or melatonin receptor agonist.

132. The EVA ring according to claim 130, wherein the EVA segment containing melatonin or melatonin receptor agonist is about 74.5 mm to about 156.0 mm in length.

133.-136. (canceled)
